(19) United States
(12) Patent Application Publication
(10) Pub. No.: US 2008/0294237 A1
(43) Pub. Date: Nov. 27, 2008

(54) INFLATABLE DEVICES AND METHODS TO PROTECT ANEURYSMAL WALL

(76) Inventor: Jack Fa-De Chu, Santa Rosa, CA (US)

Correspondence Address: JACK CHU, 5918 SUNHAWK DRIVE, SANTA ROSA, CA, 95409, US

(21) Appl. No.: 12/037,112

(22) Filed: Feb. 26, 2008

Related U.S. Application Data
(60) Provisional application No. 60/910,148, filed on Apr. 4, 2007.

(51) Int. Cl. A61F 2/82

(52) U.S. Cl. 623/1.15; 623/1.21

(57) ABSTRACT

Methods and systems for preventing aneurysm rupture and reducing the risk of migration and endoleak are disclosed. Specifically, an inflatable liner is applied directly to treat the interior of the aneurysm site. Also disclosed are methods to deliver the inflatable liner directly to treatment sites.

This application claims the benefit of U.S. Provisional Application No. 60/910,148, which was filed Apr. 4, 2007, the disclosure of which is incorporated herein by this reference.

### FIELD OF THE INVENTION

Methods and devices for preventing rupture of an aneurysm and reducing the risk of endoleak are disclosed. Specifically, methods and systems for applying inflatable multiple-layer liners directly to treatment sites and to the interior of the vessel wall are provided.

### BACKGROUND OF THE INVENTION

An aneurysm is a localized dilation of a blood vessel wall usually caused by degeneration of the vessel wall. These weakened sections of vessel walls can rupture, causing an estimated 32,000 deaths in the United States each year. Additionally, deaths resulting from aneurysmal rupture are suspected of being underreported because sudden unexplained deaths are often misdiagnosed as heart attacks or strokes while many of them may in fact be due to ruptured aneurysms.

Approximately 50,000 patients with abdominal aortic aneurysms are treated in the U.S. each year, typically by replacing the diseased section of vessel with a tubular polymeric graft in an open surgical procedure. However, this procedure was risky and not suitable for all patients. Patients who were not candidates for this procedure remained untreated and thus at risk for aneurysm rupture or death.

A less-invasive procedure is to place a stent graft at the aneurysm site. Stent grafts are tubular devices with one or more metallic stents attached to the polymeric grafts such as Dacron® or ePTFE film. The size of the tubular graft is usually matched to the diameter of the healthy vessel adjacent to the aneurysm. The metallic stent is generally stitched, glued or molded onto the biocompatible tubular covering and provides strength to the graft. In other embodiments, one or more inflatable channels were attached to the tubular graft for additional strength, and, in some cases, replaced the metal scaffold. Usually, stent grafts can be positioned and deployed at the site of an aneurysm using minimally invasive procedures. Essentially, a delivery catheter having a tubular stent graft compressed and packed into the catheter's distal tip is advanced through an artery to the aneurismal site. The tubular stent graft is then deployed within the vessel lumen in juxtaposition to the diseased vessel wall, and forming a flow conduit without replacing the dilated section of the vessel. This new flow conduit insulates the aneurysm from the body's hemodynamic forces, therefore decreasing hemodynamic pressure on the disease portion of the vessel and reducing the possibility of aneurysm rupture.

While tubular stent grafts represent improvements over more invasive surgery procedures, there are still risks associated with their use to treat aneurysms. Stent graft migration and endoleak are the biggest challenges for tubular stent grafts due to several reasons. Frequently, most of the support for the tubular stent graft depends on its fixation on a very limited section of healthy vessel between the renal artery and the aneurysm, i.e. at the neck of the aneurysm. The aneurysm sac between the aneurysm wall and the tubular stent graft is usually filled with blood or unorganized thrombosis providing little or no support to the stent graft. This vulnerable aneurysm sac is also prone to endoleak. Stent graft migration is especially common in aneurysms with short neck where there is insufficient overlap between the stent graft and the vessel, and in tortuous portions of the vessels where stent graft tends to kink resulting high hemodynamic forces on the stent graft.

Stent graft migration can break the seal between the tubular stent graft and vessel and lead to Type I endoleak, or the leaking of blood into the aneurismal sac between the outer surface of the stent graft and the inner surface of the blood vessel. This endoleak can result in the aneurysm wall being exposed to hemodynamic pressure again, thus increasing the risk of rupture.

Other than Type I endoleak, many patients experience some other issues after undergoing stent graft therapy for their aneurysms. Type II endoleak is defined as the leakage due to patent collateral arteries in the aneurismal sac. The patent collateral arteries (inferior mesenteric artery, lumbar artery) in the aneurismal sac can lead to an increased pressure in the aneurysm and may cause aneurysm enlargement and rupture in some patients. Type III and IV endoleaks are leaks caused by defects in the stent grafts. As a result, physicians often have to follow up closely with patients after endovascular therapy and perform secondary intervention to stop the leakage if it is required. Both follow-up procedures and secondary interventions are undesirable because the cost and the risk involved in those procedures.

Based on the foregoing, one goal of treating aneurysms is to provide a therapy that does not migrate or leak. To achieve this goal, stent grafts with anchoring barbs or hooks that engage the vessel wall have been developed to enhance their attachment to the wall as described in U.S. Pat. Nos. 6,395,019B2, 7,081,129B2, 7,147,661B2, 2003/0216802A1. Additionally, endostaple that punches through both graft and vessel wall to fix stent graft to the vessel wall has been developed. While these physical anchoring devices have proven to be effective in some patients, tubular stent grafts are still prone to kink. Migration and endoleaks are still reported in many patients.

Other than the improvement of the stent graft, several attempts have been made to prevent endoleak by embolizing the aneurismal sac with thrombosis or fillers such as coils, gel, fibers, etc. U.S. Pat. Nos. 6,658,288 and 6,748,953 discussed the methods to use electrical potential to create thrombosis in the aneurysm. U.S. Pat. Nos. 5,785,679, 6,231,562, 6,613,037, 7,033,389, 637,973, 6,656,214, 633,100, 6,569,190, 2003/135264A1, 36745A1, 44358A1, 2005/90804A1 and WO95/08289 disclose methods and devices to embolize the aneurismal sac. Those methods and devices create hardened material in the aneurismal sac to prevent endoleaks. However, embolization agent or dislodged emboli can travel downstream and embolize small vessels in the legs or colon. As a result, a stent graft or a barrier layer is usually utilized to exclude the aneurismal sac from the major blood conduit before injecting embolization agent into the aneurismal sac. This approach reduces the chance for the emboli to pass through the barrier layer and travel to the iliac arteries. However, the junctions to the collateral vessels in the aneurismal sac are not protected. Physicians usually will occlude the patent collateral vessels before the embolization procedure. Unfortunately, it is very difficult to identify the patency of the collateral vessels (inferior mesenteric artery, lumbar artery) in the aneurismal sac by the current imaging techniques, such as CT or MRI. If those collateral vessels are patent, i.e. a Type II endoleak is diagnosed, there is a risk that the injected embolization agent or dislodged emboli will migrate into those collateral vessels and embolize important vessels in the lumbar and colon.

Due to the risk of accidental embolization, some have proposed that the injected filler is contained in a graft or a membrane and the aneurismal sac be isolated before the injection of filler, as disclosed in U.S. Pat. Nos. 6,729,356, 5,843,160, 5,665,117, 2004/98096A1 and 2006/212112A1, which are fully incorporated by reference herein. The fill structure generally has a spherical shape, and there is typically a tubular main conduit in the middle for restoring the original geometry of the flow conduit. However, there are several concerns with this approach. First, to avoid endoleaks and migration, a close contact between the outer wall of the fill structure and the aneurysm wall is important to seal the junctions of the aorta to the origins of the collateral branch arteries. Because the fill structure is constrained by the aneurysm wall and the stent graft (or a shaping balloon) in the middle, it is essential to inject sufficient amount of filler in the fill structure to maintain close contact between the aneurysm wall and fill structure and, at the same time, avoid injecting excess amount of filler and exerting additional stress on the weak aneurysm wall. However, the gap between the fill structure and the aneurysm wall cannot be visualized easily (no contrast agent in gap or aneurysm wall) under Fluoroscope during the inflation of the fill structure, physician cannot determine if the gap has been filled (or not being filled) by the fill structure. This uncertainty can cause excess amount of filler in the fill structure and consequently high stress on the aneurysm wall and place the patient in great risk. Additionally, the aneurysm is usually sealed by a stent graft or a lumen shaping balloon before the fill structure is inflated. Existing blood in the aneurysm (with the added filler) can also cause high stress on the aneurysm wall during the inflation of fill structure if the collateral arteries in the aneurysm are occluded. Third, a significant amount of filler is required to fill the aneurismal sac for patients with large aneurysms. The effect of this large chunk of filler on vessel movement and the adjacent organs is still unknown.

Thus, there is a need to develop a new method to treat an aneurysm site to protect the aneurysm and reduce the risk of endoleak and rupture. The present invention addresses this opportunity by providing methods and systems to protect the aneurysm and to reduce the likelihood of endoleak, migration and rupture at aneurysm sites.

### SUMMARY OF THE INVENTION

The present invention addresses the issues with the current therapies by providing methods and systems to reduce the likelihood of migration, endoleak and rupture at aneurysm sites. The systems comprise an inflatable liner which is larger or the same size as the aneurysm. The inflatable liner comprises an absorbent encapsulated between a flexible outer wall and a flexible inner wall. It has a pliable mode and a strengthening mode. In its pliable mode, this inflatable liner is flexible and can be loaded into a catheter. After the liner is introduced in the aneurysm, the liner expands and conforms to the surface of the aneurysm wall. The conformation of the liner to the aneurysm wall is achieved by the flexible walls and a hemodynamic force. During the inflation of the liner, the outer wall of the liner remains in close contact with the aneurysm wall. The body fluid permeates through the flexible walls and activates the absorbent in the liner. The activated absorbent absorbs body fluid and expands the thickness of the liner. Because the outer wall is still in contact with the aneurysm wall, the inner wall of the liner moves away from the inner surface of the aneurysm in a restrained fashion by the connectors between the walls and defines the flow conduit. After deploying in the aneurysm, the body fluid transforms the liner from the pliable mode to the strengthening mode to support the aneurysm wall. The resulting strengthened liner is “locked” in the ancurysm with minimum chance to migrate out of its designated location.

In another embodiment of this invention, the inflatable liner has encapsulated absorbent that expands in large volume after picking up body fluid in the aneurysm. Many suitable absorbent can be used in the liner. The preferable absorbent is a hydrogel or a hydrophilic material which can absorb a large volume of body fluid after it is in contact with the body fluid. The absorbent can be laminated between two flexible walls by spraying, coating, dipping on the walls and dried. At least one wall of the inflatable liner is permeable to the body fluid. Before the absorbent is activated by the body fluid and expanding, the absorbent is flexible and enhances the flexibility of the inflatable liner. After the liner is deployed in the aneurysm, the body fluid passes through the wall and enables the absorbent to expand. The expanded absorbent pushes the walls outward and thus thickening and strengthening the liner. After this transformation from pliable mode to strengthening mode, the inflated liner locked in the aneurysm providing reinforcement to the aneurysm wall.

In another embodiment of this invention, inflatable liner can be fabricated with many methods. Inflatable liner can be made by joining two flexible pouch shape walls together. The space between the walls defines at least one inflatable chamber to be filled by the absorbent. Each wall can be made from the same or different material. The walls are connected by a stripe, a string or a bond, such as glue bond, weld bond, heat bond, etc. at a plurality of locations between the walls. The material used for the connector should have a significant inelasticity to avoid excess stretching during inflating. The extent of the connection can be a single point, an area, a line, or a dotted line. Combined with the walls, the arrangement and the type of connector define the inflatable chamber and are important for the flexibility of the liner. If the span of the connector between the walls is long, the liner is thick with a lower flexibility after inflation. On the other hand, if the span of the connector is short, the liner is thin with a higher flexibility at the connector. It is preferable that the liner is relatively thinner near the opening of the flow conduit to increase its flexibility to comply with patient's anatomy near the opening for optimum seal. On the other hand, the inflatable liner can be thicker in the middle of the aneurysm for additional strength and aneurysm protection.

In another embodiment of this invention, absorbent filled inflatable channels are bonded together side-by-side to form inflatable chambers of the inflatable liner. In yet another embodiment of this invention, the absorbent filled inflatable channels can be bonded to a pouch shape wall to form an inflatable liner. In another embodiment of this invention, an inflatable liner is formed by attaching a plurality of inflatable patches on either side of a pouch shape wall. The space between the inflatable patch and the wall is filled by absorbent.

In yet another embodiment according to the present invention, a bioactive or a pharmaceutical agent is incorporated into the liner. The bioactive or pharmaceutical agent can be mixed with the absorbent before laminating in the liner. After deploying in the aneurysm, the bioactive or pharmaceutical agent diffuses into the aneurysm wall and treats the damage in the vessel. Because the liner of this invention is in close contact with the aneurysm wall, the bioactive or pharmaceutical agent can reach the aneurysm wall without being diluted by the blood if the agent is delivered systematically by injection. Many bioactive or pharmaceutical agents can be used to treat aneurysm. Drugs that inhibit matrix metalloproteinases, inflammation or other pathological processes involved in aneurysm progression, can be incorporated into the absorbent to enhance wound healing and/or stabilize and possibly reverse the pathology. Drugs that induce positive effects at the aneurysm site, such as growth factor, can also be delivered with the absorbent and the methods described herein. Alternatively, the bioactive or pharmaceutical agent can be coated on the outer surface of the inflatable liner directly against the aneurysm wall.

In another embodiment of the present invention, the surface of the liner is treated with a fibril, coating, foam or surface texture enhancement. These coatings or surface treatment can increase the surface area on the outer wall of the liner and promote tissue or cell to grow onto the outer surface of the liner. The attached cells or tissue on the liner can enhance the bonding and seal between the vessel wall and the liner. In addition to enhanced bonding, appropriate surface coating or texture can also promote the formation of thrombosis and increase the seal between the liner and the aneurysm wall.

In yet another embodiment of this invention, a plurality of inflatable liners can be deployed sequentially in an aneurysm to increase their protection on the aneurysm. Several inflatable liners can be used in the same aneurysm to increase the total thickness of the liners. Alternatively, inflatable liners of different constructions can be used to achieve the optimum liner performance. For example, inflatable liner facing the aneurysm wall can comprise a more porous outer surface with a better tissue attachment to the aneurysm wall. On the other hand, the inflatable liner facing the flow conduit can comprise more absorbent that resulting a stiffer liner with a better support to the flow conduit.

In another embodiment of the present invention, the inflatable liner is particularly suitable for lining aneurysm with some distance from the bifurcation, especially abdominal aortic aneurysms (AAA) with some distance from the iliac bifurcation. The walls of the liner are flexible with three openings. The space between the outer and inner walls defines at least one inflatable chamber filled by absorbent. One or more connectors between the walls define the thickness of the inflatable chamber and the liner. The inner wall of the liner determines the blood flow conduit with one inlet and two outlets. After deployed in the aneurysm, the liner would have a shape defined by the inner surface of the aneurysm. The blood flow conduit would have a shape determined by the inner surface of the aneurysm, connector and the thickness of the liner.

In another embodiment of the present invention, the inflatable multiple walls liner is particularly suitable for lining aneurysm close to the bifurcation, especially abdominal aortic aneurysms (AAA) adjacent to the iliac bifurcation. The walls of the liner are flexible with three openings. The space between the outer and inner walls defines at least one inflatable chamber filled by absorbent. One or more connectors between the walls define the thickness of the inflatable chamber and the liner. The inner wall of the liner determines the blood flow conduit with one inlet and two outlets. After deployed in the aneurysm, the liner would have the shape defined by the inner surface of the aneurysm. The blood flow conduit would have a shape determined by the inner surface of the aneurysm and the thickness of the liner.

In another embodiment of the present invention, the inflatable liner is particularly suitable for lining aneurysm which has extended from aorta to the iliac artery. The walls of the liner are flexible with a bifurcation and two sleeves. The space between the outer and inner walls defines at least one inflatable chamber filled by the absorbent. One or more connectors between the walls define the thickness of the inflatable chamber and the liner. The inner wall defines the blood flow conduit with one inlet and two outlets. After deployed in the aneurysm, the liner would have the shape defined by the inner surface of the aneurysm. The blood flow conduit would have a shape determined by the inner surface of the aneurysm and the thickness of the liner.

In yet another embodiment of the present invention, the systems to treat aneurysm also include at least one stent which is placed near the opening of the liner after the liner is deployed in the aneurysm. Preferably, the stent is deployed at the junction between the liner and the vessel wall to ensure no gap between them. Usually, the stent is most useful to be deployed at the inlet of the blood flow conduit. Optionally, stent can be deployed at the outlet of the blood flow conduit. Alternatively, portion of the stent can be covered with a graft or a membrane to further assist the sealing between the liner and vessel wall. Alternatively, one or more stents can be fixed to the liner by sewing, stitching, glue bond, weld bond, heat bond, etc.

In the practice, physician needs to determine the appropriate liner to use in each patient. Through the imaging techniques such as CT scan or MRI, the size and length of the patient's aneurysm can be measured accurately. Then, the physician can select a liner that best fit the patients' aneurismal anatomy. It is preferred to use a liner with outer diameter no less than the largest inner diameter of the aneurysm. Because the flexible walls of the liner and the hemodynamic force in the liner, the liner will remain conform to the inner surface of the aneurysm.

For a preferred deployment method of this invention, a multi-lumen catheter with an expandable element is used to deliver the inflatable liner in the aneurysm. The expandable element has a first configuration and a second configuration. The first configuration allows the expandable element to be compressed into the catheter for delivery with minimum invasivity. The second configuration allows the expandable element to expand and anchor the liner at the proximal end of the aneurysm. Additionally, the expandable element on the multi-lumen catheter is configured to allow blood perfusion through the expandable element at the second configuration. Many expandable elements, such as balloon, stent, etc. can be used in this invention. An annual shape balloon is used herein as an example. In its pliable mode, portion of the inflatable liner near the inlet is placed on top of the balloon with its inner surface against the balloon. After the inflatable liner and balloon are both collapsed into the low profile configurations, they can be compressed and loaded into a sheath on the catheter and sterilized with various known sterilization methods. Then, the liner delivery system can be positioned in the aneurysm site via iliac artery with minimum invasivity. It is preferable that the balloon on the distal end of the catheter is deployed near the neck of the aneurysm to ensure no excess stress is applied on the aneurysm. After the balloon is deployed, portion of the inflatable liner near the inlet is pressed against the vessel wall by the inflated balloon. At the same time, blood flows through the lumen in the balloon, and the hemodynamic force expands the liner radially toward the aneurysm wall. As the sheath is retrieved to expose the liner in the sheath, the expansion continues until the liner covers the whole inner surface of the aneurysm. This procedure is safe because the hemodynamic force to expand the liner is the same hemodynamic force existed in the aneurysm before the operation. No additional stress is placed on the aneurysm wall during the expansion of the liner. After the inner surface of the aneurysm is completely covered by the liner, a second expandable element (e.g. proximal balloon) is inflated at the junction between the liner and the vessel. This proximal balloon can be on the same multi-lumen catheter or on a separate one. The purpose of this proximal balloon is to ensure the patency of blood flow conduit during the inflation of liner. The transition of the liner from pliable mode to the strengthening mode gives addition strength to the liner and protects the aneurysm. It is accomplished by the expansion of the absorbent and the liner after the absorbent absorbs the body fluid in the aneurysm. As the liner is inflated, the status of inflating is monitored by the radiopaque markers on the liner. Alternatively, a radiopaque agent is incorporated into the liner so that the whole liner is visible under fluoroscope. Because the liner is already conformed to the inner surface of the aneurysm, the transformation “locks” the inflated liner in the aneurysm against the aneurysm wall. Then the balloons are collapsed, and the delivery catheter is retrieved from the patient's body. Optionally, a stent or a membrane covered stent is placed at junction between the liner and the vessel wall to ensure seal.

In another deployment method of this invention for treating patient with aneurysm close to the bifurcation (iliac artery), a multi-lumen catheter is used to deliver a stent attached liner in the aneurysm. Expandable element such as a distal balloon can be used in this particular deployment method. The distal balloon is positioned near the distal end of the multi-lumen catheter. In the collapsed configuration, a distal stent and a portion of the liner is placed on top of the distal balloon. After the stent attached liner is collapsed into a low profile configuration, it is compressed and loaded into a sheath in the multi-lumen catheter and sterilized. Then the catheter/liner system can be positioned in the aneurysm site via the iliac artery with minimum invasivity. It is preferred that the distal stent is deployed near the neck of the aneurysm to ensure no excess stress is applied on the aneurysm. After the distal stent is deployed, portion of the liner is pressed against the vessel wall by the deployed stent. Then, the sheath of the catheter is removed to expose the to-be expanded liner. The liner expands radially toward the aneurysm wall by a hemodynamic force and eventually conforms to the inner surface of the aneurysm wall. After the inner surface of the aneurysm wall is completely covered by the liner, both iliac stents are deployed in iliac arteries respectively to ensure seal at junctions between the liner and iliac arteries. Then a balloon catheter is inserted in the liner via the left iliac artery. Once it is in position, a second balloon on the distal end of the balloon catheter is inflated with saline. At about the same time, a proximal balloon on the delivery catheter is also inflated by saline. Both balloons are used to ensure patency of the flow conduit when the liner is inflating. The transition of the liner from pliable mode to strengthening mode gives addition strength to the liner and protects the aneurysm. It is accomplished by the inflation of the liner after the absorbent picks up the body fluid in the aneurysm. As the liner is inflating, the status of inflation is monitored by radiopaque markers on the liner. Because the outer wall of the liner is already conformed to the inner surface of aneurysm wall, the inner wall of liner moves away from aneurysm wall during the inflation. This transition to strengthening mode also “locks” the liner in the aneurysm against migration. Finally, all balloons are deflated, and the delivery catheter is retrieved from the patient's body leaving the inflated liner in aneurysm. This invention is particularly suitable for treating patients with abdominal aortic aneurysms near the iliac bifurcation.

In another embodiment according to the present invention, a bioactive or a pharmaceutical agent is incorporated into the liner. The bioactive or pharmaceutical agent can be mixed with the absorbent. After the deployment of liner in the aneurysm, the agent diffuses into the aneurysm wall and treats the damage in the vessel. Because the liner of this invention is in close contact with the aneurysm wall, the agent can reach the aneurysm wall without being diluted by the blood if the agent is delivered systematically by injection. Many bioactive or pharmaceutical agents can be used to treat aneurysm. Drugs that inhibit matrix metalloproteinases, inflammation or other pathological processes involved in aneurysm progression, can be incorporated into the filler to enhance wound healing and/or stabilize and possibly reverse the pathology. Drugs that induce positive effects at the aneurysm site, such as growth factor, can also be delivered with the filler and the methods described herein. Alternatively, the bioactive or pharmaceutical agent can be coated on the outer surface of the liner directly against the aneurysm wall.

In another embodiment of the present invention, the surface of the liner is treated with fibril, coating, foam or surface texture enhancement. These coatings or surface treatment can increase the surface area on the outer wall of the liner and promote tissue or cell to grow onto the outer wall of the liner. The attached cells or tissue on the wall can enhance the bonding and seal between the vessel wall and the liner. In addition to enhanced bonding, appropriate surface coating or texture can also promote the formation of thrombosis and therefore increase the seal between the liner and the aneurysm wall.

There are several benefits to treat aneurysm with this present invention. 1. The inflatable multiple walls liner strengthens the aneurysm wall and prevents the rupture of aneurysm by reducing the hemodynamic pressure on the aneurysm wall. 2. The collapsed liner is flexible so that it can be loaded in a catheter and access the aneurysm site with minimum invasivity. 3. The flexibility of the liner and the hemodynamic force allow the liner to conform to the inner surface of the aneurysm wall. After the liner is strengthened, it will be “locked” in the aneurysm without endoleak or migration. 4. Less material is required to cover the inner surface of the aneurysm wall. The resulting liner is more flexible and compatible with the vessel and adjacent organs. 5. There is no excess amount of stress on the vulnerable aneurysm wall during the deployment of the liner. In order to prevent endoleak and migration, it is essential to have close contact between the outer wall of the liner and the surface of the aneurysm wall. This invention addresses the drawbacks of prior arts and allows the liner to conform to the aneurysm wall without placing excess stress on the fragile aneurysm wall. As a result, the systems and methods provided by this present invention are safer than methods disclosed in prior arts. 6. The flexible liner does not have the issue of kinking or occlusion of blood flow which is common in tubular stent graft. 7. The durability of the liner is better than the tubular stent graft because there is no untreated space, which is prone to endoleak between the liner and aneurysm wall. 8. The present invention can enhance the adhesion of the liner to the aneurysm wall further reducing the risk of liner migration and endoleak. 9. This invention enables the use of bioactive or pharmaceutical agents in the liner to treat aneurysm without dilution. The pathological processes involved in aneurysm progression can be stabilized and possibly be reversed.

### BRIEF DESCRIPTION OF THE FIGURES

FIG. 1a depicts the perspective view of an inflatable liner as described in one embodiment according to the present invention.

FIGS. 1b-c depict the cross sectional views of the inflatable liner as described in FIG. 1a.

FIGS. 2a-b depict the enlarged cross sectional views of the inflatable liner as described in FIG. 1a.

FIG. 2c depicts the enlarged cross sectional view of an inflatable liner as described in one embodiment according to the present invention.

FIG. 3a depict the perspective view of an inflatable liner as described in one embodiment according to the present invention.

FIGS. 3b-c depict the cross sectional views of the inflatable liner as described in FIG. 3a.

FIGS. 4a-b depict the enlarged cross sectional views of the inflatable liner as described in FIG. 3a.

FIG. 4c depicts the enlarged cross sectional view of an inflatable liner as described in one embodiment according to the present invention.

FIG. 5 depicts the perspective view of an inflatable channel as described in one embodiment according to the present invention.

FIG. 6 depicts the perspective view of an inflatable channel as described in one embodiment according to the present invention.

FIG. 7a depicts the exterior view of an inflatable liner as described in one embodiment according to the present invention.

FIGS. 7b-c depict the cross sectional views of the inflatable liner as described in FIG. 7a.

FIGS. 8a-b depict the cross sectional views of an inflatable liner as described in one embodiment according to the present invention.

FIGS. 9a-e depict the exterior views of inflatable liners as described in embodiments according to the present invention.

FIG. 10a depicts the perspective view of an inflatable liner as described in one embodiment according to the present invention.

FIG. 10b depicts the cross sectional view of the inflatable liner as described in FIG. 10a.

FIG. 11a depicts the perspective view of an inflatable liner as described in one embodiment according to the present invention.

FIG. 11b depicts the cross sectional view of the inflatable liner as described in FIG. 11a.

FIGS. 12a-e depict the perspective views of inflatable liners as described in embodiments according to the present invention.

FIG. 13a depicts the perspective view of the delivery catheter as described in one embodiment according to the present invention.

FIG. 13b depicts the collapsed inflatable liner on the delivery catheter as described in one embodiment according to the present invention.

FIGS. 14a-h depict deploy sequence of an inflatable liner in the aneurysm sac according to the teachings of the present invention.

FIGS. 15a-h depict an alternate method to deploy an inflatable liner in the aneurysm sac according to the teachings of the present invention.

FIGS. 16a-j depict another alternate method to deploy an inflatable liner in the aneurysm sac according to the teachings of the present invention.

### DETAILED DESCRIPTION

Embodiments according to the present invention provide inflatable liner and methods useful for protecting aneurysm and reducing the risk of implantable medical device post-implantation migration and endoleak. More specifically, the inflatable liner and methods provide protection to blood vessel against rupture especially at the aneurysm site. The inflatable liner also has the advantage of no kinking, minimizing post-implantation device migration and endoleak following liner deployment at an aneurismal site.

For convenience, the devices, compositions and related methods according to the present invention discussed herein will be exemplified by using inflatable multiple walls liner intended to treat abdominal aorta aneurysms or Thoracic aortic aneurysms. However, aneurysms at other locations of the body can be treated with the same devices or methods.

The present invention addresses the issues with current therapies by providing methods and systems to reduce the likelihood of kinking, migration, endoleak and rupture at aneurysm sites. The systems offer a device to protect the aneurysm from hemodynamic force and leave no vulnerable gap between the device and the aneurysm wall. These systems comprise an inflatable liner which is larger than or the same size as the aneurysm to be treated. It has an outer and an inner walls with encapsulated absorbent between the walls. At least one of the walls is permeable to the body fluid. This liner has both pliable mode and strengthening mode. In the pliable mode, this inflatable liner is flexible and can be compressed and loaded in a delivery catheter. After the liner is placed at the aneurysm site, the flexible liner expands under a hemodynamic force and conforms to the inner surface of the aneurysm without a gap. This close contact with aneurysm wall is important because it allows no vulnerable “gap” or “space” between the liner and the weak aneurysm wall which is prone to endoleak. After deploying in the aneurysm, the inflatable liner transforms from the pliable mode to the strengthening mode by the activated absorbent in the liner. The body fluid in the aneurysm permeates through the wall of the liner to activate and expand the absorbent encapsulated in the liner, therefore resulting in a thicker and stiffer liner to strengthen the aneurysm wall. Because the inflatable liner is conforming to the usually complex topography of the inner surface of the aneurysm, the inflated liner is “locked” in the aneurysm with minimum chance for migrating out of its designated location providing reinforcement to the weak aneurysm wall. The inner wall of the inflated liner defines the blood flow conduit.

The close contact with aneurysm wall is important for the inflatable liner because any gap between the liner and the weak aneurysm wall is prone to endoleak. To achieve this, the liner has to be flexible in the pliable mode so that it can conform to the complex topography of the inner surface of aneurysm. The liner also has to be larger than or the same size as the aneurysm so that the liner can expand plastically by a hemodynamic force and conform to the inner surface of the aneurysm without a gap between them. Other than the properties of the liner, the deploying method is important to achieve conformation to the aneurysm wall. The hemodynamic force to expand the liner has to be sufficient to expand the liner without causing excess stress on the aneurysm wall. Additionally, the existing blood in the aneurysm has to be drained from the aneurysm while the liner is expanding. The details of the deploying method will also be disclosed in this invention.

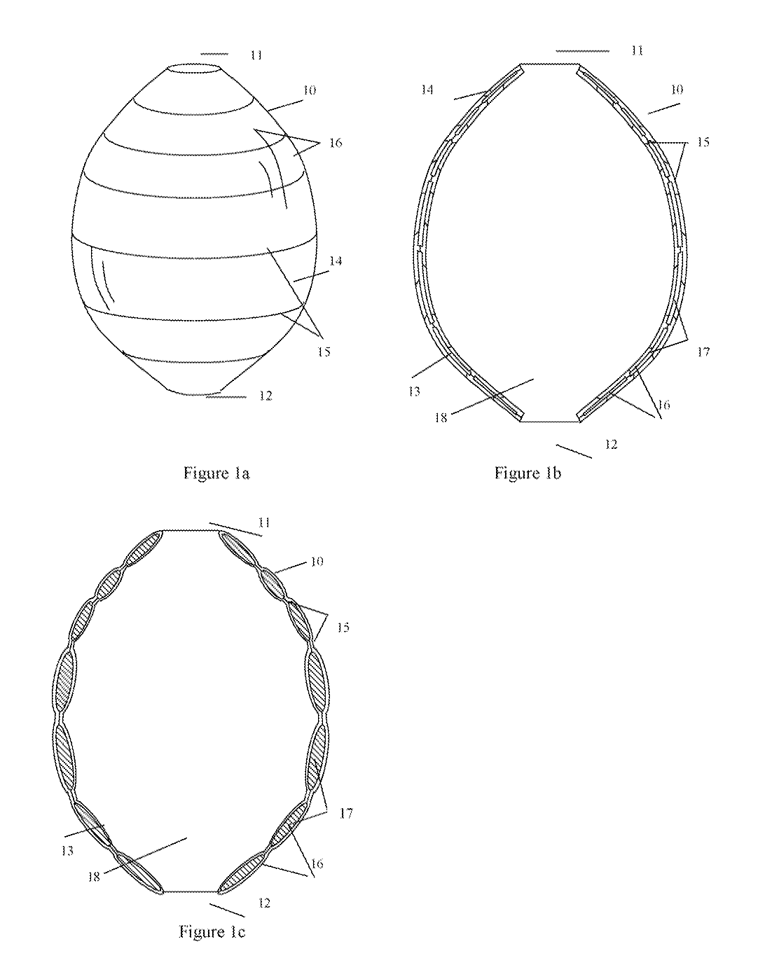

In the present invention, as illustrated in FIG. 1a, inflatable liner 10 has the general appearance of a hollow pouch with two openings 11, 12. FIG. 1b shows the cross sectional view of liner 10 with inner wall 13 and outer wall 14. Connectors 15 link inner wall 13 and outer wall 14 together at various locations to form interconnected inflatable chambers 16 between the walls 13, 14. Absorbent 17 is encapsulated in inflatable chambers 16. Flow conduit 18 is defined by inner wall 13 and two openings 11, 12. As shown in FIG. 1c, absorbent 17 expands inflatable chambers 16 after it is activated by the body fluid (not illustrated). The embodiment of this invention with two openings 11, 12 is particularly suitable for treating patients with Thoracic aortic aneurysm (TAA), aneurysms in the peripheral arteries, or abdominal aortic aneurysms (AAA) with some distance from the iliac bifurcation.

Absorbent 17 can be laminated between walls 13, 14 by spraying, coating, dipping and dried. Those processing techniques are well known to the people skilled in the art. A radiopaque agent such as barium sulfate or gold powder can also be included in absorbent 17 or embedded in walls 13, 14 to enhance the visibility of liner 10 under fluoroscopy. To avoid premature expansion, liner 10 and delivery catheter have to remain dry before use. At least one of walls 13, 14 is permeable to the body fluid. Before absorbent 17 is activated by the body fluid and expands, absorbent 17 is flexible and enhances the flexibility of liner 10. In its pliable mode, liner 10 is flexible and can be compressed and loaded in a delivery catheter. After the delivery catheter has reached the aneurysm site, the flexibility of liner 10 enables it to expand radially and conform to the aneurysm wall by a hemodynamic force. After liner 10 is deployed in the aneurysm, the body fluid passes through walls 13, 14 and enables absorbent 17 to absorb the body fluid and expand. The expanded absorbent 17 pushes walls 13, 14 outward and thus thickening and strengthening liner 10 as shown in FIG. 1c. At this moment, liner 10 transforms into strengthening mode and is locked in the aneurysm to prevent migration and provide reinforcement to the weak aneurysm wall.

Many different methods can be used to connect two walls together in the present invention. Some exemplifiable connectors are a stripe, a string or a direct bond, such as glue bond, weld bond, heat bond, etc. Each inflatable liner can utilize one particular connector or a mix of several different type connectors to achieve the desired performance. The type of connector also determines the thickness of the liner after inflation. If a stripe or a string is used, its span between the walls defines the thickness of the liner. The material used for the connector can be the same material used for the walls with significant inelasticity to avoid excess stretching during inflation. If a heat bond is used as the connector, the thickness of the walls becomes the thickness of the liner at the bonding. The extent of the connection by the bonding between the walls can be a single point, an area, or a line. Combined with the walls, the arrangement and the type of connection between the walls define the configuration of the inflatable chamber filled by the absorbent. As illustrated in FIG. 1b, inner wall 13 and outer wall 14 are bonded together circumferentially between two openings 11, 12 to form a plurality of inflatable chambers 16 filled by absorbent 17.

The materials used for walls 13, 14 are biocompatible and flexible so that walls 13, 14 can conform to the inner surface of the aneurysm. According to the teaching of the present invention, walls 13, 14 can be constructed with sheets or films. Each wall 13, 14 can be made from the same or a different biocompatible material. Typical biocompatible materials are Dacron®, Nylon, PET, PE, PP, polyurethane, ethylene vinyl acetate, FEP or ePTFE. They can be extruded, weaved, blow molded or molded into thin sheet or film. The processing technologies are well known to person specialized in film or sheet processing. The thin sheet or film is stitched, glued, bonded or directly molded into the desired shape. Or they can be made by spraying, coating, and dipping, etc. polymer solution directly on a mold and dried. At least one of walls 13, 14 is permeable to the body fluid. It can be fabricated by creating holes or pores on the walls by laser, by punctuation or by salt leaching process, etc. The techniques to manufacturing permeable sheets are known to people in this art.

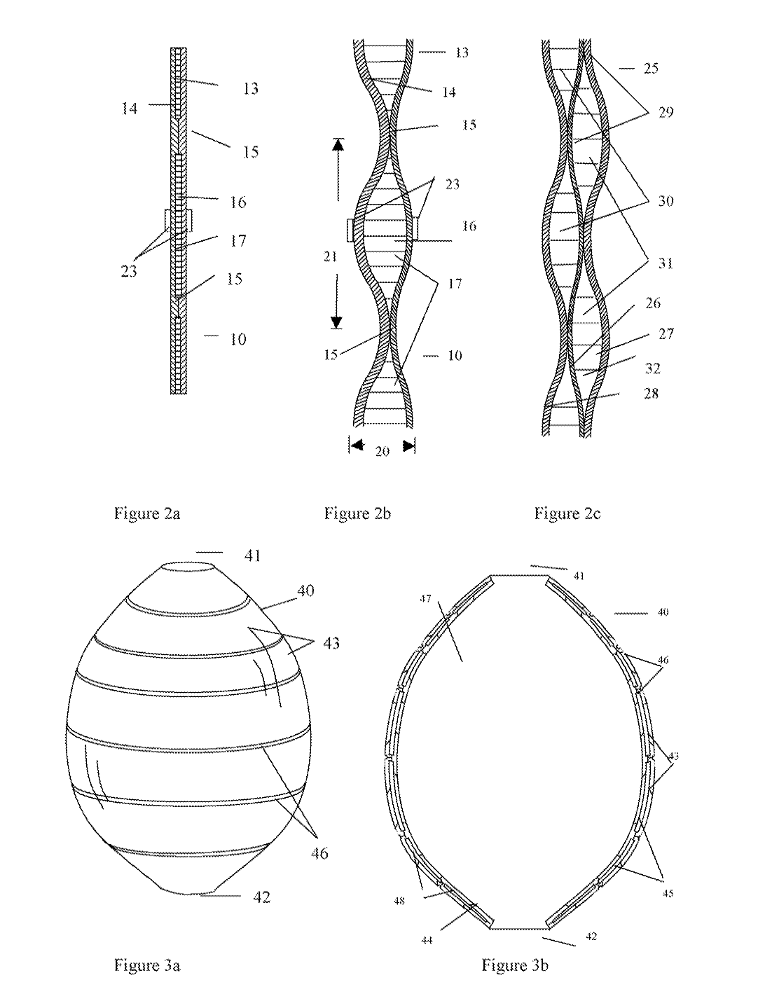

FIGS. 2a-b are the enlarged cross sectional views of walls 13, 14 in the exemplifiable inflatable liners 10 (in FIGS. 1a-c) according to the teaching of this invention. In FIG. 2a, outer wall 14 of liner 10 is bonded to inner wall 13 at connectors 15 forming an inflatable chamber 16 with encapsulated absorbent 17. FIG. 2b describes the cross sectional configuration of liner 10 after inflatable chamber 16 is inflated by the activated absorbent 17. Various bonding techniques such as glued bond, weld bond, heat bond, etc. can be used at a plurality of locations between walls 13, 14. The extent of bond can be a dot, an area, a line or a dotted line or a combination of above.

Span 20 of inflatable chamber 16 between walls 13, 14 is one of the factors affecting the flexibility of liner 10 as illustrated in FIG. 2b. If span 20 is long, inflatable chamber 16 is thick with a lower flexibility after inflation. If span 20 is short, inflatable chamber 16 is thin with a higher flexibility after inflation. Liner 10 is usually thinner at places where connector 15 joins walls 13, 14. The thinner liner 10 at connector 15 forms a “soft point” enabling liner 10 to bend easier at that location and accommodate body's movement in the aneurismal. As a result, those connectors 15 can be placed in locations where more flexibility is needed. Additionally, distance 21 between connectors 15 is another factor affecting the flexibility of liner 10. Liner 10 is thinner at connectors 15 and becomes thicker away from connectors 15. If distance 21 is long, inflatable chamber 16 would be thick with a lower flexibility after inflation because walls 13, 14 are not constrained by connector 15 and expand outwards by the activated absorbent as illustrated in FIG. 2b. If distance 21 is short, inflatable chamber 16 is thin with a higher flexibility after inflation. In this invention, it is preferable that inflatable chamber 16 is thinner near openings 11, 12 of main flow conduit 18. This will increase the liner's flexibility to comply with patient's anatomy near openings 11, 12 to achieve the optimum seal. On the other hand, inflatable chamber 16 or liner 10 can be thicker in the middle of the aneurysm for additional strength. The thicker inflatable chamber 16 or liner 10 can be achieved by a longer span 20 or a longer distance 21 between connectors 15.

As discussed before, the aneurysm is usually weak and prone to rupture, it is critical to be able to monitor the progress of liner deployment to achieve success treatment on the aneurysm. Radiopaque markers 23 are placed on both inner 13 and outer 14 walls of liner 10 as shown in FIG. 2a-b. As the liner 10 is inflated by the activated absorbent 17, the distance between radiopaque markers 23, which can be measured under the fluoroscope, is increasing until the pre-determined liner thickness is reached. This embodiment of the present invention provides physicians a safe tool to know directly the status of the liner deployment and inflation without “guessing”.

According to the teaching of this invention, more than two walls can be used to form the liner as shown in the cross sectional configuration of liner 25 in FIG. 2c. A third wall 26 is laminated between inner 27 and outer 28 walls. Alternating bonds 29 between these walls 26, 27, 28 create a plurality of inflatable chambers 30, 31 between walls 26, 27, 28. Inflatable chambers 30, 31 formed by multiple walls 26, 27, 28 can be filled by the same absorbent 32 or absorbent of various hardness to achieve the optimum protection on the aneurysm wall. For example, inflatable chambers 30 closer to outer wall 28 can be filled with a softer absorbent for better conformation with the aneurysm wall. Inflatable chambers 31 closer to inner wall 27 can be filled with a harder absorbent for better support for flow conduit. Alternatively, inflatable chambers 30 closer to outer wall 28 are filled with less absorbent resulting in a lower inflating pressure after the absorbent is activated. The lower pressure results in softer inflatable chambers 30 for better conformation with the aneurysm wall. On the other hand, inflatable chambers 31 closer to inner wall 27 are filled by more absorbent resulting in stiffer chambers 30 for better support for flow conduit.

According to the teaching of this invention, many suitable absorbent can be used in the liner. The preferable absorbent is a biocompatible hydrogel or a hydrophilic material which can absorb a large volume of fluid and expand in volume after it is in contact with the body fluid. The fluid can be blood, water, etc. Exemplary non-limiting biocompatible hydrogel or hydrophilic material includes hydrogel, polymethacrylic acid, polyacrylic acid, polyesters, polyacrylamide, polyacrylamide copolymer, sodium acrylate and vinyl alcohol copolymer, polyvinyl alcohol, polyacetals, polyvinyl acetate, acrylic acid ester copolymer, polyvinyl pyrrolidone, polyacrylonitrile, polyarylethernitriles, Hypan, poly(2-hydroxyethyl methacrylate)(polyHEMA), Carbomer copolymer and homopolymer, alkoxylated surfactants, polyethylene oxide, poly(propylene oxide), poly(ethylene glycol), poly(propylene glycol), poly(vinylcarboxylic acid), collagen, polyvinyl pyridine, polylysine, polyarginine, poly aspartic acid, poly glutamic acid, polytetramethylene oxide, methoxylated pectin gels, cellulose acetate phthalate, gelatin, alginate, calcium alginate, Carbopol, Poloxamer, Pluronic, Tetronics, PEO-PPO-PEO triblocks copolymer, Tetrafunctional block copolymer of PEO-PPO condensed with ethylenadiamine, Poly(acrylic acid) grafted (PEO-PPO-PEO-PAA) copolymers, graft copolymers of Pluronic and poly(acrylic acid), alkylcellulose, hydroxyalkylcellulose, PEG-PLA-PEG block polymers, Poly(N-isopropylacrylamide) (PNIPAAm), tetrafunctional block copolymer of PEO-PPO-ethylenadiamine, copolymer of PNIPAAm and acrylic acid (AAc), P(NIPAAm-co-AAc), copovidone, povidone, Hyaluronic Acid (HA), polyoxyalkylene ether, cellulose acetate, cellulose acetate butyrate, cellulose diacetate, nitrocellulose, starch, and the mixture or copolymer of above. The preferable hydrogels disclosed in present invention are polyacrylamine, polyacrylic acid and polyvinyl pyridine. The hydrogel or hydrophilic materials can be fabricated in forms of powder, solution, foam, mesh, fibrils, slurry, gel, etc with a high flexibility before absorbing body fluid.

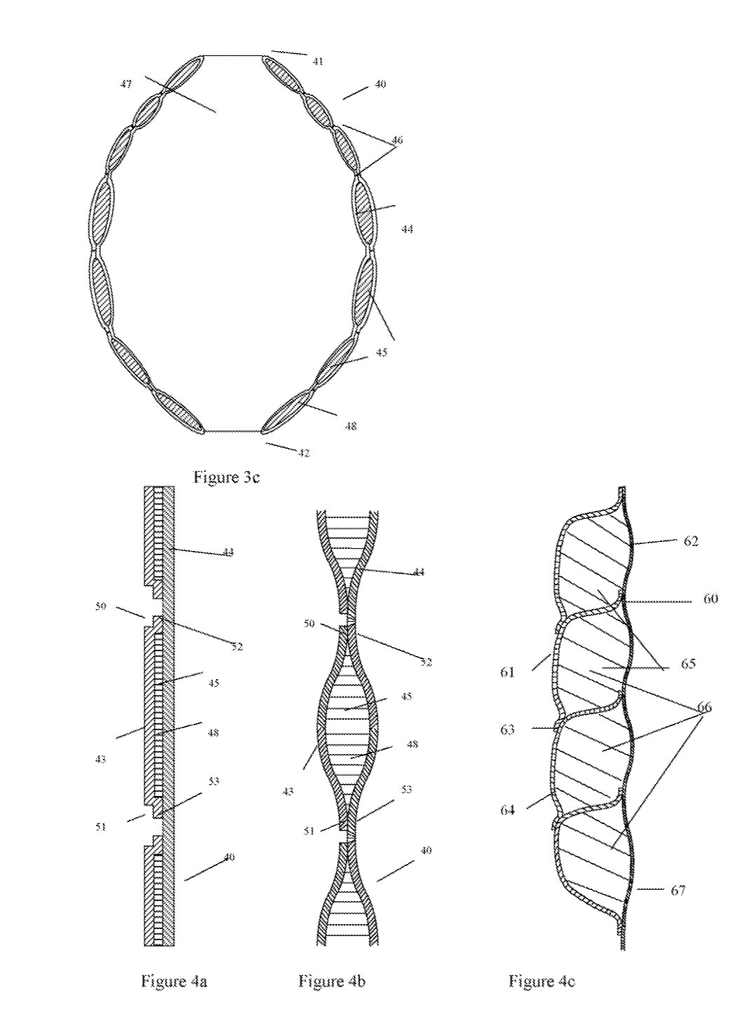

In another embodiment of this invention, the inflatable liner is formed by attaching a plurality of inflatable patches on a pouch shape wall. FIG. 3a illustrates an exemplary inflatable liner 40 with two openings 41, 42. Inflatable patches 43 can be connected to either side of the pouch shape wall 44. In this example, inflatable patches 43 are connected to the outside of wall 44 circumferentially between two openings 41, 42 herein to form inflatable liner 40. Other than circumferential pattern, various connector patterns can be used to bond inflatable patch 43 to wall 44. As shown in the cross sectional view of liner 40 in FIG. 3b, inflatable patches 43 are bonded to pouch shape wall 44 forming inflatable chambers 45 in liner 40. The bond between patch 43 and pouch shape wall 44 are connectors 46. Each inflatable chamber 45 is defined by inflatable patch 43, wall 44 and connectors 46. As illustrated in FIG. 3b, wall 44 defines blood flow conduit 47 with a first opening 41 and a second opening 42. After deployment in the aneurysm, body fluid in the aneurysm permeates through inflatable patch 43, wall 44 and is absorbed by absorbent 48 in inflatable chamber 45. The activated absorbent 48 expands inflatable chamber 45 and the thickness of liner 40 transforming liner 40 from the pliable mode to the strengthening mode as illustrated in FIG. 3c. Inflated liner 40 would have the shape defined by the inner surface of the aneurysm. Blood flow conduit 47 would have a shape determined by the inner surface of the aneurysm and the thickness of inflated liner 40. This embodiment of the present invention is particularly suitable for treating patients with Thoracic aortic aneurysm (TAA), aneurysms in the peripheral arteries, or abdominal aortic aneurysms (AAA) with some distance from the iliac bifurcation.

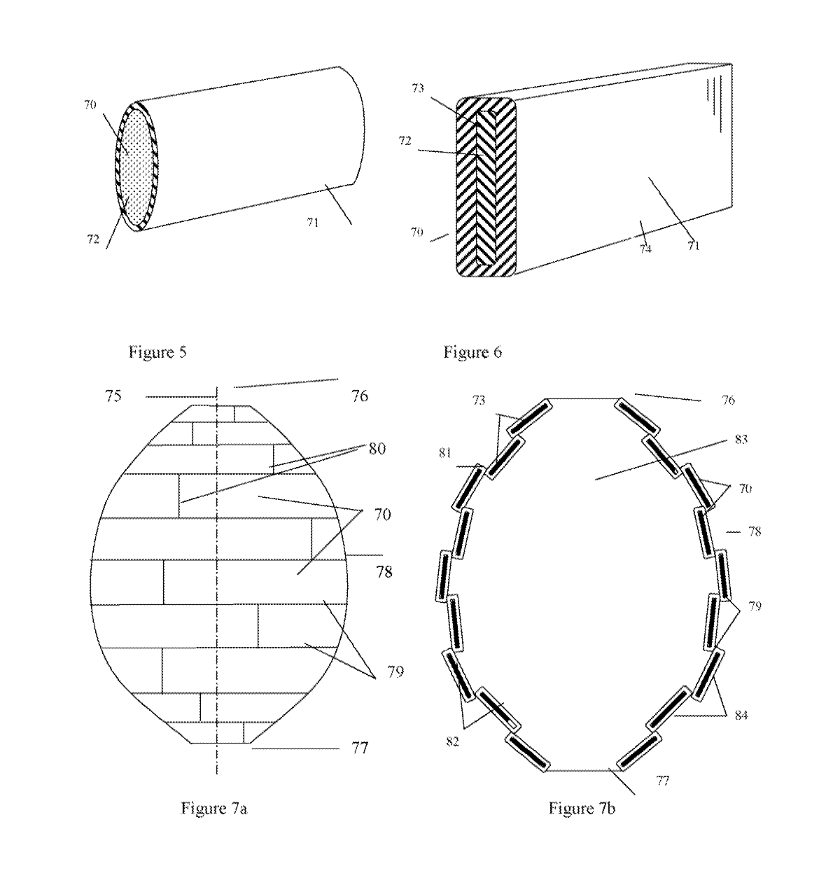

FIGS. 4a-b are the enlarged cross sectional views of liner 40 in FIGS. 3a-c, inflatable chamber 45 is formed by bonding two edges 50, 51 of inflatable patch 43 on pouch shape wall 44. As a result, inflatable chamber 45 and the encapsulated absorbent 48 are defined by inflatable patch 43, wall 44 and two connectors 52, 53. The biocompatible material used for inflatable patch 43 and wall 44 is permeable to the body fluid (not shown). The attachment of inflatable patch 43 on wall 44 can be performed by glue bond, weld bond, heat bond, etc. After the absorbent 48 is activated by the body fluid, absorbent 48 absorbs the body fluid and expands outwards to increase the thickness of inflatable chamber 45 and liner 40 as depicted in FIG. 4b. Alternatively, inflatable patches 43 can be attached on either side of pouch shape wall 44. Alternatively, inflatable patch can be placed on top of adjacent patch. As depicted in FIG. 4c, while one edge 60 of patch 61 is bonded to pouch shaped wall 62, the other edge 63 of patch 61 is bonded to the adjacent patch 64 forming an inflatable chamber 65 with encapsulated absorbent 66. Patches 61, 64 become outer wall of liner 67, and pouch shape wall 62 becomes the inner wall. A portion of patches 61, 64 becomes connectors between pouch shape wall 62 and patches 61, 64. After the absorbent is activated, a relatively consistent liner thickness can be achieved by this approach.

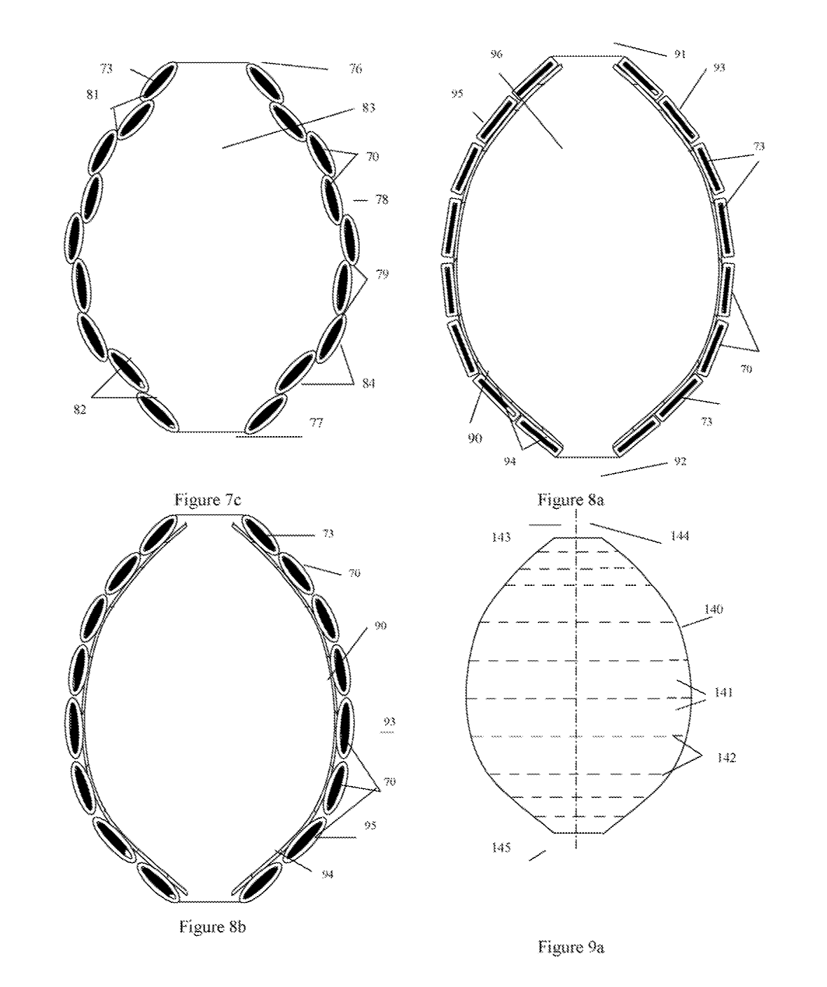

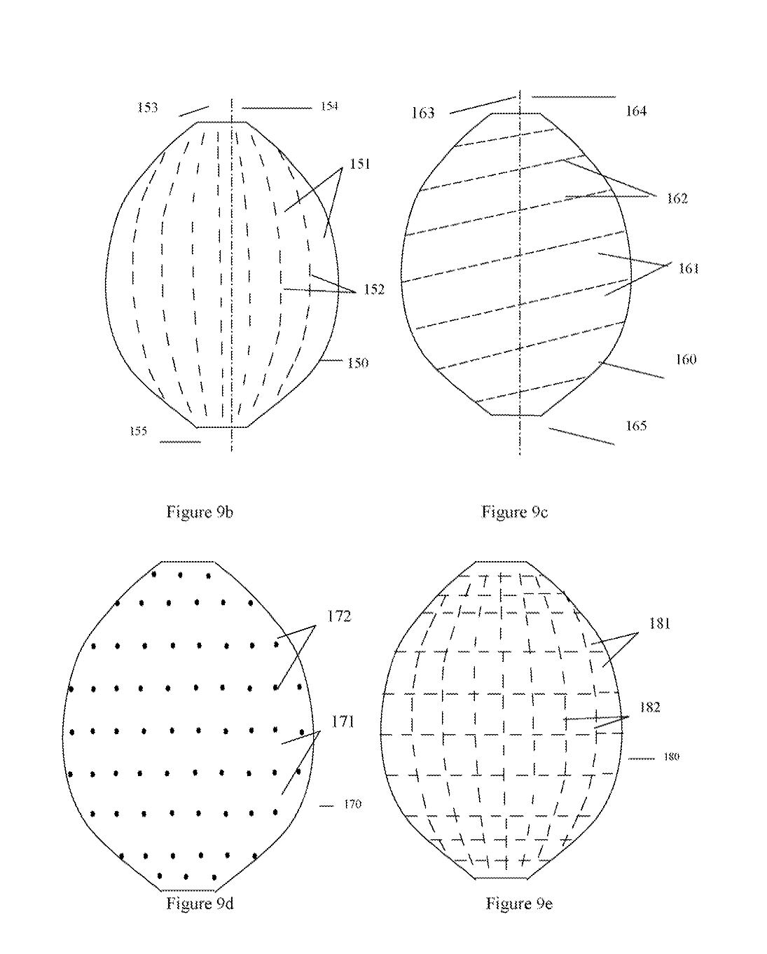

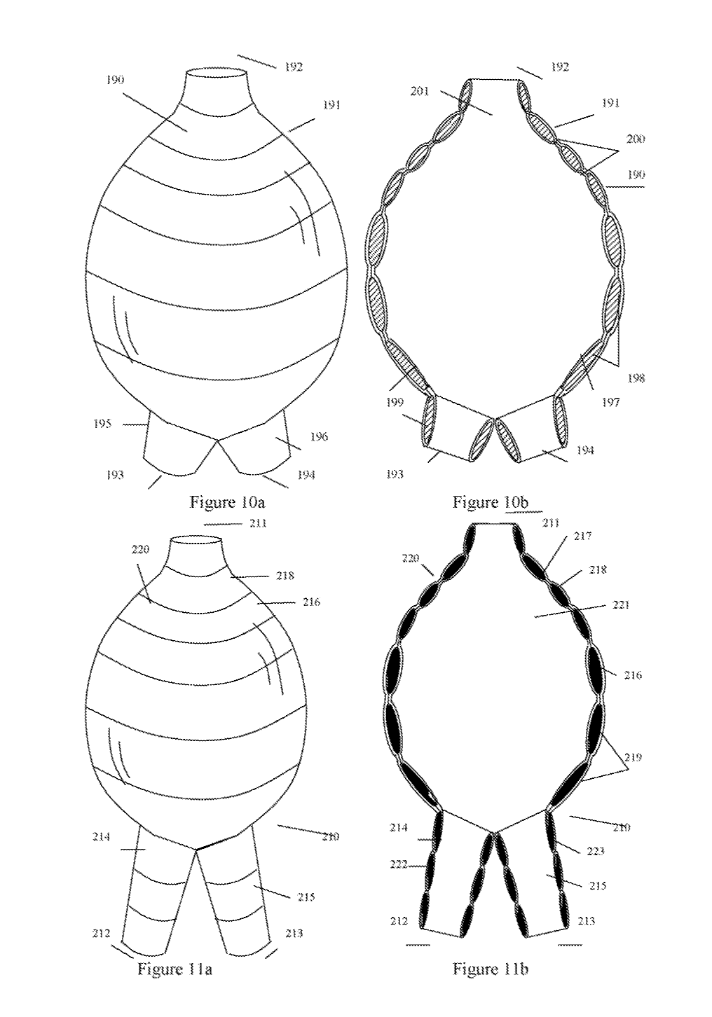

In another embodiment of this invention, inflatable channels are bonded together to form the inflatable liner. As shown in FIG. 5, inflatable channel 70 comprises flexible wall 71 which is permeable to body fluid. Inflatable channel 70 also comprises inflatable chamber 72 which is filled with absorbent 73. Then absorbent 73 filled inflatable channel 70 is flattened to form stripe 74 as shown in FIG. 6. A plurality of inflatable channels 70 are connected together side-by-side circumferentially around the axis 75 between two openings 76, 77 to form inflatable liner 78 as illustrated in FIG. 7a. The pattern of connectors 79 on liner 78 can affect the flexibility and strength of inflatable liner 78. The circumferential connector pattern described herein is one of the exemplifiable patterns according to the teaching of this invention. Ends 80 of inflatable channel 70 are sealed to form a discontinuous loop as illustrated in FIG. 7a. As shown in the cross sectional view of liner 78 in FIG. 7b, absorbent 73 filled inflatable channels 70 are connected together side-by-side at edges 81 of inflatable channel 70. Inner wall 82 defines blood flow conduit 83 with a first opening 76 and a second opening 77. After deploying in the aneurysm, body fluid (not shown) in the aneurysm permeates through inner wall 82, outer wall 84 and is absorbed by absorbent 73 in inflatable channel 70. The activated absorbent 73 expands inflatable channel 70 and the thickness of liner 78 transforming liner 78 from the pliable mode to the strengthening mode as illustrated in FIG. 7c. As discussed above, connectors 79 creates a thinner area in liner 78 and serves as a “stress relief” enhancing the flexibility of liner 78 in the axial direction. In the aneurysm, liner 78 would have the shape defined by the inner surface of the aneurysm. Blood flow conduit 83 would have a shape determined by the inner surface of the aneurysm and the thickness of liner 78. This invention is particularly suitable for treating patients with Thoracic aortic aneurysm (TAA), aneurysms in the peripheral arteries, or abdominal aortic aneurysms (AAA) with some distance from the iliac bifurcation.

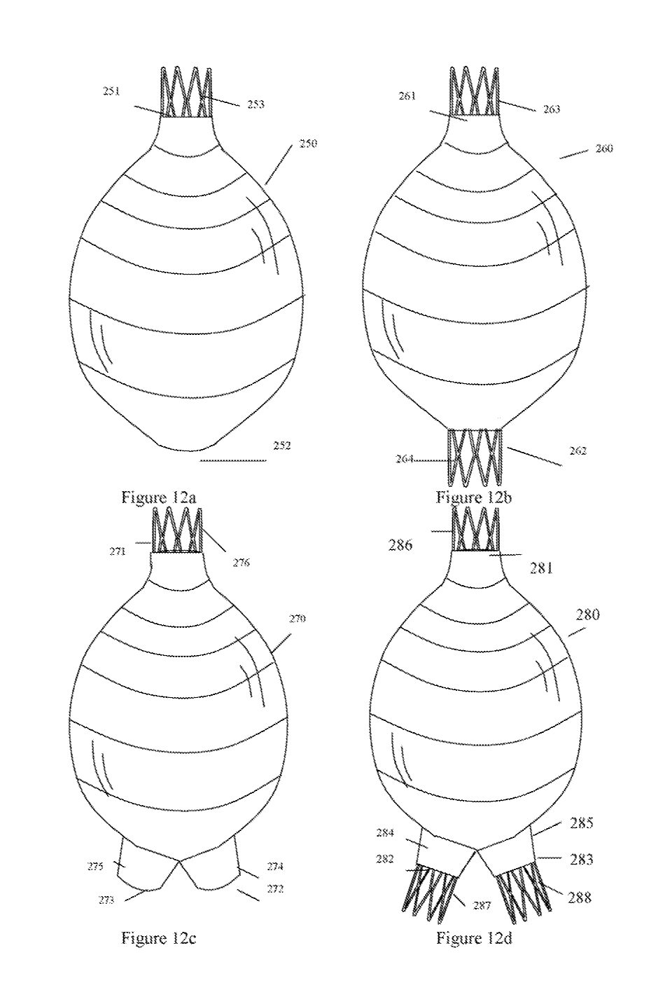

In another embodiment of this invention, absorbent 73 filled inflatable channels 70 are connected to a pouch shape wall 90 circumferentially between two openings 91, 92 to form liner 93. The prospective view of liner 93 would be similar to the inflatable liner 78 described in FIG. 7a. FIGS. 8a-b are the cross sectional views of the exemplifiable wall configurations of liner 93. Absorbent 73 filled inflatable channels 70 are connected to pouch shape wall 90 with inner wall 94 and outer wall 95 as shown in FIG. 8a. The connection of inflatable channels 70 on wall 90 can be done by glue bond, weld bond, heat bond, etc. Blood flow conduit 96 is defined by inner wall 94. As depicted in FIG. 8b, inflatable channels 70 expands outwards to increase the thickness of liner 93 after absorbent 73 in liner 93 is activated by the body fluid (not shown). Alternatively, inflatable channels 70 can be bonded to either side of pouch shape wall 90 to form a liner.

In another embodiment of the present invention, an inflatable multiple walls liner is created by combining inflatable chambers of various forms such as an inflatable patch and an inflatable channel.

As discussed above, the span of connector between the walls and the distance between the connectors determine the thickness and flexibility of the inflatable chamber and liner. Direct bonding between the walls forms a relatively short connector (i.e. the span is merely the thickness of the bond) with thin liner at the bonding. A shorter distance between the connectors with a shorter connector span leads to a liner with a thinner wall. On the other hand, a longer distance between the connectors with a longer connector span (in the case of using connector such as a strip or a wire) results in a thicker liner. As a result, the thickness and flexibility of the liner can be controlled by selecting the appropriate connector, the distance between the connectors and the connector span between the walls.

Additionally, the arrangement, (i.e. pattern), of connectors in the liner is also important in determining the flexibility and strength of the liner. The pattern defines not only the distance between the connectors but also the orientation of the connectors. As discussed above, connectors may result in a thinner area in the liner and serve as a “soft point” for the liner. This characteristic allows the liner to have flexibility in the desired direction to conform to body movement. At the same time, it is also desirable to have a liner with sufficient thickness and strength to protect the aneurysm from rupturing.

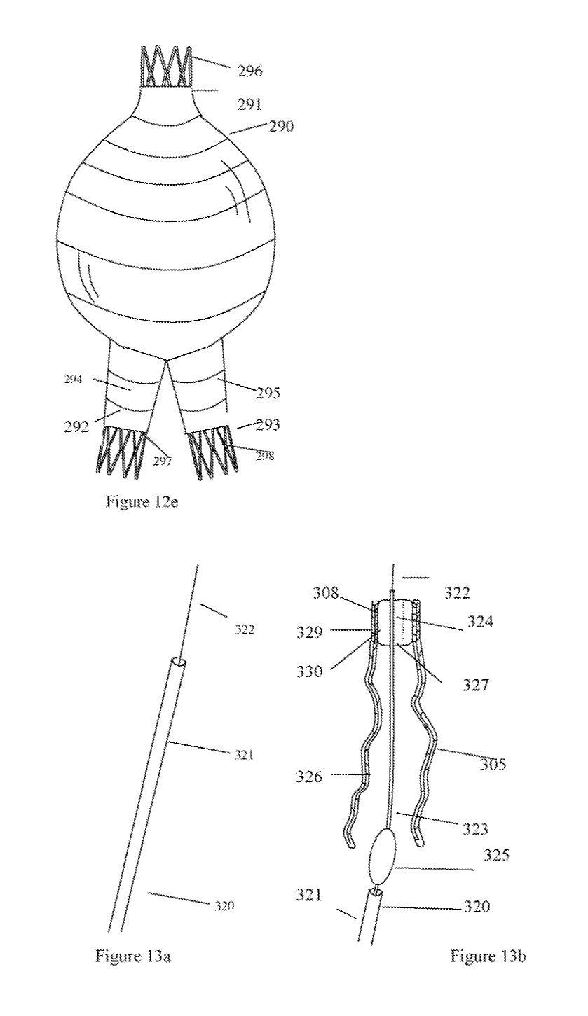

Some exemplary connector patterns are described in FIGS. 9a-e. The dotted lines or points indicate the locations of the connectors in the wall. A strip, a string, a direct bonding or a combination of the foregoing can be utilized to form one or more connectors between the walls. The walls and connectors define inflatable chambers in the respective liners with which they are illustrated. A plurality of flow ducts (not shown) between inflatable chambers allow fluid communication between inflatable chambers in the liners.

As shown in FIG. 9a, inflatable multiple walls liner 140 comprises plurality of inflatable chambers 141 (divided by connectors 142) arranged circumferentially along axis 143 between two openings 144, 145. This connector pattern provides liner 140 with a high flexibility along axis 143 between two openings 144, 145 and a high circumferential stiffness after liner 140 is inflated. On the other hand, liner 150, shown in FIG. 9b, has plurality of inflatable chambers 151 (divided by connectors 152) arranged along axis 153 between two openings 154, 155. Due to its connector pattern, liner 150 has a high flexibility circumferentially and a high stiffness along axis 153 after it is inflated. FIG. 9c illustrates a liner 160 with inflatable chambers 161 (divided by connectors 162) encircling axis 163 helically between two openings 164, 165. This particular connector pattern has a compromised flexibility and stiffness as compared to liners 140 and 150 in both circumferential and axial directions after liner 160 is inflated.

Liners 170 and 180 with connector patterns described in FIGS. 9d-e do not have a particular stiffness or flexibility bias in either circumferential or axial direction. Actually, there is only one inflatable chamber 171 with a plurality of pointed connectors 172 in liner 170 described in FIG. 9d. FIG. 9e illustrates liner 180 with inflatable chambers 181 (divided by connectors 182) with no particular stiffness or flexibility bias in either circumferential or axial direction.

In another embodiment of the present invention, a connector is placed at a needed location to serve as “stress relief” or a “bend point” because of the thinner liner near the connector as discussed above. The circumferential flexibility of liner 140 described in FIG. 9a can be enhanced by introducing connectors in the axial direction as shown in FIG. 9b. These exemplary connector patterns are described herein to demonstrate the ability to achieve a desirable liner flexibility and stiffness by utilizing various connectors, and by varying their orientation, distance between connectors and thickness.

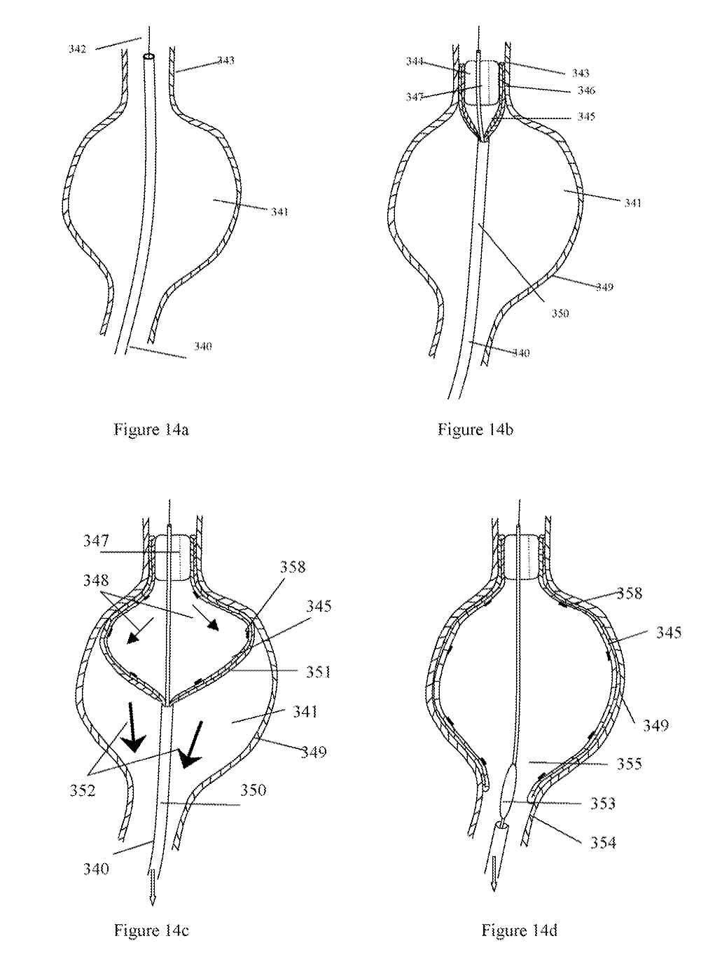

In another embodiment of the present invention, the inflatable liner is particularly suitable for lining an aneurysm disposed in close proximity to a bifurcation, such as an aortic aneurysm adjacent to the iliac artery. FIGS. 10a-b are the perspective and cross sectional views of the exemplary liners according to the teaching of this invention. In FIG. 10a, outer wall 190 of liner 191 is flexible with three openings 192, 193 and 194. Two openings 193, 194 leading to the bifurcation are adjacent to each other. There are sleeves 195, 196 connected to openings 193, 194 respectively to enhance the seal between liner 191 and the vessel wall. The space between outer wall 190 and inner wall 197 comprises at least one inflatable chamber 198 filled by absorbent 199 as depicted in the cross sectional view of liner 191 in FIG. 10b. Pluralities of connectors 200 between walls 190, 197 determine the thickness of inflated liner 191. A short span connector (e.g. connector formed via bonding) is used herein as an example. However, a long span connector (e.g. a connector formed via strip or string) can also be used. Inner wall 197 defines blood flow conduit 201 with one inlet 192 and two outlets 193, 194. Each of the outlets 193, 194 leads to an iliac artery respectively. After the deployment within the aneurysm, liner 191 will have the shape defined by the morphology of the inner surface of the aneurysm wall. The shape of blood flow conduit 201 will be determined by both the morphology of the inner surface of the aneurysm wall and the thickness of liner 191.

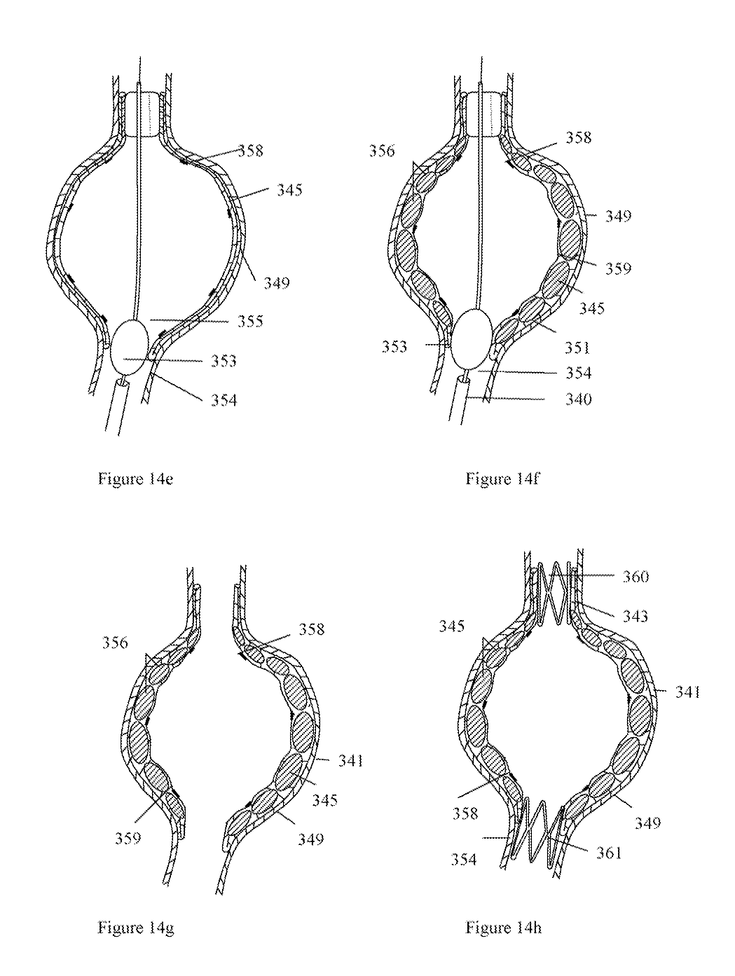

In yet another embodiment of the present invention, the inflatable liner is particularly suitable for lining aneurysm which has extended from aorta to iliac artery. FIGS. 1a-b are the perspective and cross sectional views of the exemplary liners according to the teaching of this invention. Liner 210 is hollow with three openings 211, 212, 213 as shown in FIG. 11a. Two of the openings 212, 213 leading to the bifurcation are adjacent to each other and are configured to mate with an iliac artery respectively. Sleeves 214, 215 extended from openings 212, 213 enhance the seal between liner 210 and the vessel wall and protect aneurysm in the iliac arteries. The space between outer wall 216 and inner wall 217 comprises at least one inflatable chamber 218 filled by absorbent 219 as depicted in the cross sectional view of liner 210 in FIG. 1b. Pluralities of connectors 220 between walls 216, 217 define the thickness of inflated liner 210. A short span connector 220 (i.e. one formed via bonding) is used herein as an example. However, a long span connector (i.e. one formed via a strip) can also be used. Inner wall 217 defines blood flow conduit 221 with one inlet 211 and two outlets 212, 213. Inflatable bifurcated sleeves 214, 215 have inflatable chambers 222 and 223 providing protection to the aneurysms in both iliac arteries. After deployment within the aneurysm, blood flow conduit 221 will have a shape determined by both the inner surface of the aneurysm and the thickness of liner 210.

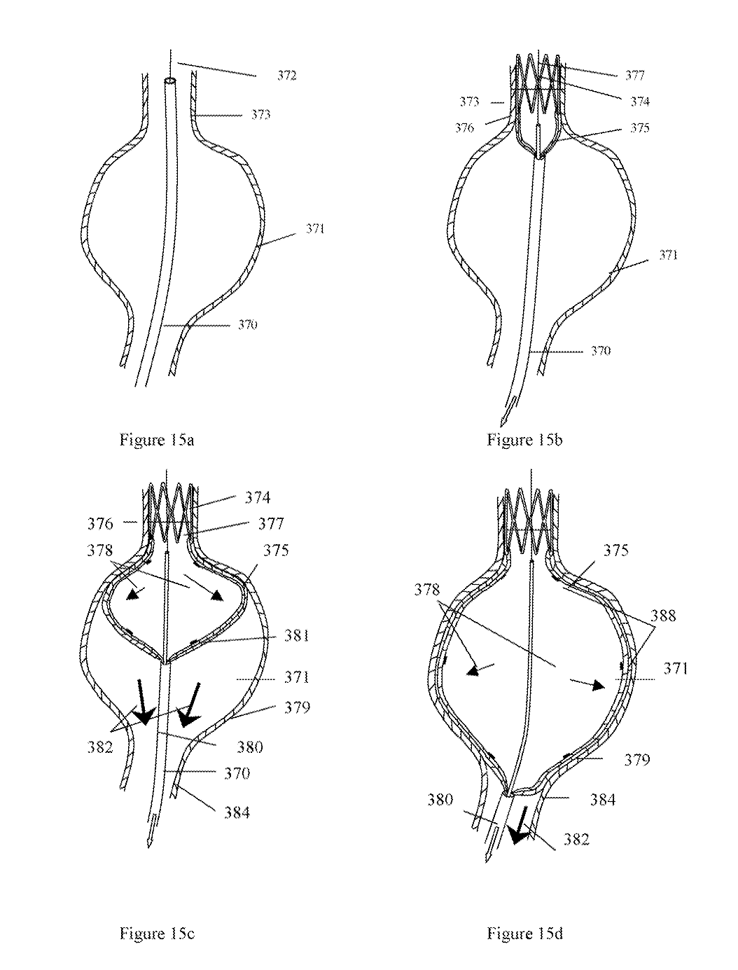

In yet another embodiment of the present invention, at least one stent is permanently fixed to one of the openings of the inflatable liner for anchoring and sealing the liner on the vessel wall. The stent is either self-expandable either or by the outward radial force exerted by another expandable element so that stent can expand and anchor liner to the vessel walls after deployment. Typical biocompatible materials for stent are stainless steel, Nitinol or plastic. FIGS. 12a-e are the perspective views of the exemplary liners according to the teaching of this invention. As shown in FIG. 12a, liner 250 is hollow with two openings 251, 252. At least one stent 253 is permanently fixed to liner 250 near opening 251. Stent 253 is stitched, glued, or bonded to inflatable liner 250. Alternatively, inflatable liner 260 is hollow with two openings 261, 262 as illustrated in FIG. 12b. One stent 263 is permanently fixed to liner 260 near opening 261. Another stent 264 is permanently fixed to liner 260 near opening 262. Stents 263, 264 are stitched, glued, or bonded to inflatable liner 260. This embodiment of the present invention is particularly suitable for treating patients with Thoracic aortic aneurysm (TAA), aneurysms in the peripheral arteries, or abdominal aortic aneurysms (AAA) with some distance from the iliac bifurcation.

As shown in FIG. 12c, liner 270 is hollow with three openings 271, 272, 273. Two of the openings 272, 273 leading to the bifurcation have sleeve 274, 275 adjacent to each other. Stent 276 is permanently fixed to liner 270 near opening 271 by stitch, glue, or heat bonding. Alternatively, liner 280 is hollow with three openings 281, 282, 283 as illustrated in FIG. 12d. Two of the openings 282, 283 leading to the bifurcation have sleeve 284, 285 adjacent to each other. Stent 286 is permanently fixed to liner 280 near opening 281. One stent 287 is permanently fixed to sleeve 284 leading to one of the iliac arteries. Another stent 288 is permanently fixed to sleeve 285 leading to the other one of the iliac arteries. This embodiment of the present invention is particularly suitable for treating patients with aneurysms adjacent to bifurcation.

Liner 290 is hollow with three openings 291, 292, 293 as shown in FIG. 12e. Two of the openings 292, 293 leading to the bifurcation have sleeves 294, 295 adjacent to each other. Each of the openings 292, 293 is configured to mate with an iliac artery respectively. Sleeves 294, 295 extended from the openings 292, 293 enhance the seal between the liner 290 and the vessel wall and protect aneurysm in the iliac arteries. Stent 296 is permanently fixed to liner 290 near opening 291. Stents 297, 298 are stitched, glued, or bonded to sleeves 294, 295 leading to iliac arteries respectively. This embodiment of the present invention is particularly suitable for treating patients with aneurysms extended from aorta to iliac artery.

In the practice, physician needs to determine the appropriate liner to use for each patient. With the imaging techniques such as CT scan or MRI, the size and length of the patient's aneurysm can be measured accurately. Then, the physician can select the inflatable multiple walls liner that best fits the patient's aneurysmal anatomy. It is preferable to use a liner with an outer diameter no less than the largest inner diameter of the aneurysm. Because of the flexible wall of the liner and the hemodynamic force, the liner will conform to the inner wall of the aneurysm. By selecting a liner with a larger diameter than the inner diameter of the aneurysm, the extra length of the liner wall will ensure conformation to the aneurysm wall with no gaps between the liner and aneurysm wall.

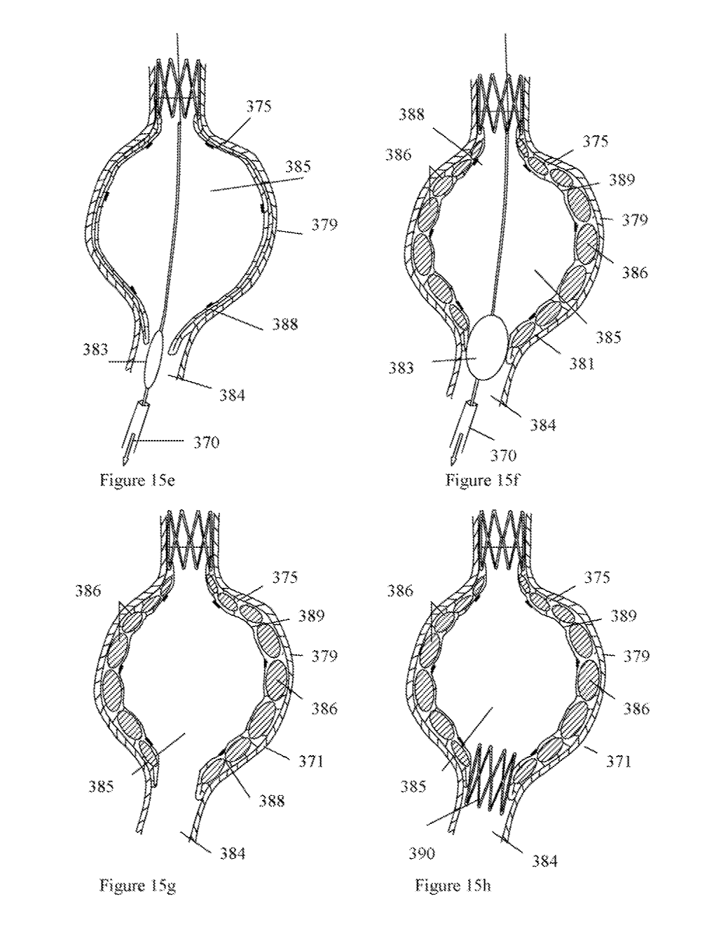

In one embodiment according to the present invention, an inflatable multiple walls liner is pre-loaded into a delivery catheter such as that depicted in FIG. 13a. Delivery catheter 320 has a retractable sheath 321 with compressed liner (not shown) in it. Guidewire 322 can pass through a lumen (not shown) in delivery catheter 320 and is used to direct delivery catheter 320 in patients' body. Within the lumen of catheter 320 is a multilumen catheter 323, as shown in FIG. 13b. Multilumen catheter 323 has a lumen for guide wire 322, lumens for delivery of saline for inflating distal balloon 324 and proximal balloon 325. Distal balloon 324 is positioned at the distal end of multilumen catheter 323 to anchor liner 326 during the deployment procedures. Other than distal balloon 324, various types of expandable elements, such as a self-expandable stent, wire, mesh, etc. can also be used to anchor liner 326 according to the invention. An inflatable distal balloon 324 is used herein as an example. Inflatable distal balloon 324 is preferred to have an annular shape with lumen 327 allowing blood flow through balloon 324 after inflation. In the pliable mode, a portion of liner 326 near inlet 329 is mounted on top of distal balloon 324 with inner wall 330 against the surface of distal balloon 324. Optionally, a second expandable element, such as proximal balloon 325, is placed near the proximal end of multilumen catheter 323. During assembly, after liner 326 and balloons 324 and 325 are collapsed into the low profile configurations, they are radially compressed to fill sheath 321 in the distal end of delivery catheter 320. Liner 326 is covered with retractable sheath 321 and sterilized with various known sterilization methods.

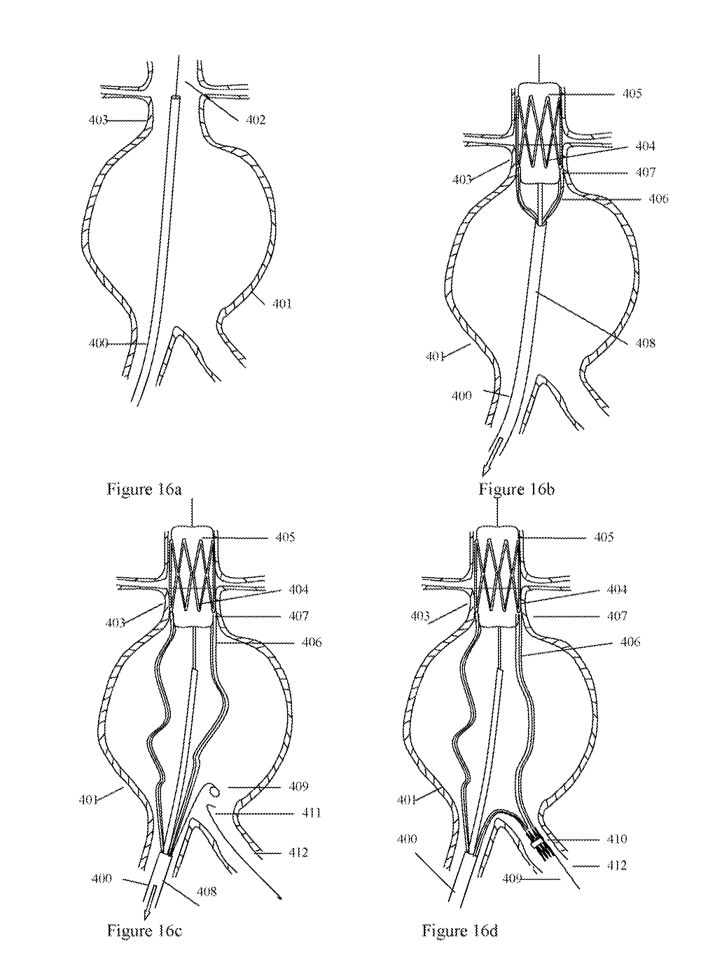

For a preferred deployment method of this invention, a multi-lumen balloon catheter 340 is used to deliver the inflatable liner in aneurysm 341 via the iliac artery using a minimally invasive technique. An inflatable liner with two openings (as shown in FIG. 1a) is used herein as an example to line aneurysm 341. As shown in FIG. 14a, delivery catheter 340 is guided by guidewire 342 and positioned in the aneurysm 341 with its distal end close to neck 343 of aneurysm 341. It is preferable that distal balloon 344 is deployed near neck 343 of aneurysm 341 to ensure that no excess stress is exerted upon aneurysm 341 as illustrated in FIG. 14b. After distal balloon 344 is inflated, a portion of liner 345 is pressed against vessel wall 346 by the inflated distal balloon 344. At the same time, blood flows through lumen 347 within distal balloon 344 (as indicated by arrow 348) to expand liner 345 radially toward aneurysm wall 349. As sheath 350 is retrieved to expose liner 345 in sheath 350, the expansion continues until outer wall 351 of liner 345 is against aneurysm wall 349 of aneurysm 341 as depicted in FIGS. 14c-d. As indicated by arrows 352 in FIG. 14c, the existing blood in aneurysm 341 escapes from aneurysm 341 through the gap between catheter 340 and aneurysm wall 349. This procedure is safe because the hemodynamic pressure to expand liner 345 is the same hemodynamic pressure that existed in aneurysm 341 before treatment. No additional stress is placed on aneurysm wall 349 during the liner expansion. After aneurysm wall 349 has been completely covered by liner 345, a proximal balloon 353 is inflated at junction 354 between liner 345 and aneurysm wall 349 as shown in FIG. 14c. Proximal balloon 353 is also preferably of an annular shape and can be on the same catheter 340 or on a separate catheter. Proximal balloon 353 is to ensure the patency of blood flow conduit 355 remains at junction 354 during the inflation of liner 345. The activation of absorbent 356 and the inflation of liner 345 give additional strength to liner 345 and protects aneurysm wall 349 as shown in FIG. 14f. As liner 345 is inflated, the status of inflation is monitored by radiopaque markers 358 on the surface of liner 345. Alternatively, the status of inflation can be observed if absorent 356 becomes radiopaque when additional radiopaque agent has been added to it. Because outer wall 351 of liner 345 already conforms to the inner surface of aneurysm wall 349, absorent 356 is actually moving inner wall 359 of liner 345 away from aneurysm wall 349. Finally, balloons 344 and 353 are collapsed, and delivery catheter 340 is retrieved from the patient's body leaving inflated liner 345 in aneurysm 341 as shown in FIG. 14g. Optionally, stents 360, 361 or, alternatively, membrane covered stents are placed between liner 345 and aneurysm wall 349 at neck 343 and junction 354 respectively to ensure adequate seal as shown in FIG. 14h.

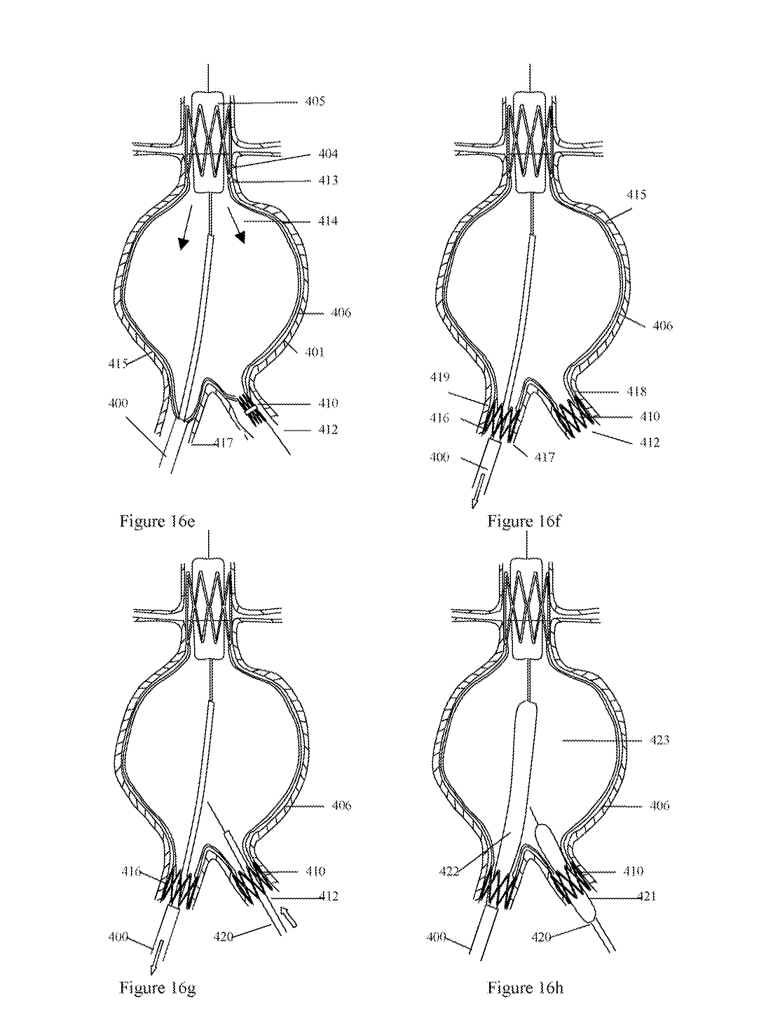

For another preferred deployment method of this invention, a multi-lumen catheter 370 is used to deliver a stent attached inflatable liner in the aneurysm 371 via the iliac artery with minimum invasivity. An inflatable liner with a self expandable stent affixed to one of its openings (as shown in FIG. 12a) is used herein as an example to line aneurysm 371. A balloon expandable stent can also be used. As shown in FIG. 15a, delivery catheter 370 is guided by guidewire 372 and positioned in aneurysm 371 with its distal end close to neck 373 of aneurysm 371. It is preferable that distal stent 374 is deployed near neck 373 of aneurysm 371 to ensure that no excess stress is exerted upon aneurysm 371 as illustrated in FIG. 15b. After distal stent 374 is deployed, a portion of liner 375 is pressed against vessel wall 376 by the deployed stent 374. At the same time, blood flows through lumen 377 in distal stent 374, as indicated by arrows 378, in order to expand liner 375 radially toward aneurysm wall 379. As sheath 380 is retrieved to expose liner 375 in sheath 380, the expansion continues until outer wall 381 of liner 375 is against aneurysm wall 379 of aneurysm 371 as depicted in FIGS. 15c-d. As indicated by arrows 382 in FIGS. 15c-d, the existing blood in aneurysm 371 escapes from aneurysm 371 through the gap between catheter 370 and aneurysm wall 379. This procedure is safe because the hemodynamic pressure to expand liner 375 is the same hemodynamic pressure that existed in aneurysm 371 before treatment. No additional stress is placed on aneurysm wall 379 during the liner expansion. After aneurysm wall 379 has been completely covered by liner 375, a proximal balloon 383 is inflated at junction 384 between liner 375 and aneurysm wall 379 as shown in FIGS. 5e-f. Proximal balloon 383 is preferably on the same catheter 370 or on a separate catheter. Proximal balloon 383 is to ensure the patency of blood flow conduit 385 at junction 384 during the inflation of liner 375. The activation of absorbent 386 and inflation of liner 375 gives additional strength to liner 375 and protects aneurysm wall 379 as shown in FIG. 15f. As liner 375 is inflated, the status of inflation is monitored by radiopaque markers 388 on the surface of liner 375. Alternatively, the status of inflation can be observed if absorbent 386 becomes radiopaque when additional radiopaque agent has been added to it. Because outer wall 381 of liner 375 already conforms to the inner surface of aneurysm wall 379, absorbent 386 is actually moving inner wall 389 of liner 375 away from aneurysm wall 379. Finally, balloon 383 is collapsed, and delivery catheter 370 is retrieved from the patient's body leaving inflated liner 375 in aneurysm 371 as shown in FIG. 15g. Optionally, stent 390 or, alternatively, membrane covered stent is placed at junction 384 to ensure adequate seal as shown in FIG. 15h.

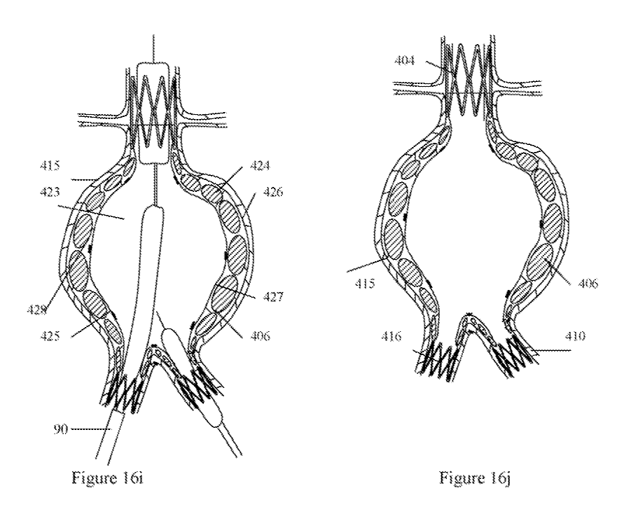

For yet another preferred deployment method of this invention, multi-lumen delivery catheter 400 is used to deliver the stent attached inflatable multiple walls liner in aneurysm 401 via the iliac artery with minimum invasivity. An inflatable multiple walls liner with three stents affixed to its three openings (as shown in FIG. 12d) is used herein as an example to line aneurysm 401 close to the bifurcation. Other exemplary stent attached inflatable multiple walls liners can also be deployed with this method. As shown in FIG. 16a, delivery catheter 400 is guided by guidewire 402 and positioned in aneurysm 401 with its distal end close to neck 403 of aneurysm 401. It is preferable that distal stent 404 is deployed by a distal balloon 405 near neck 403 of aneurysm 401 to ensure that no excess stress is exerted upon aneurysm 401 as illustrated in FIG. 16b. A balloon expandable stent 404 is used herein as an example. Other types of stent such as self expandable stent can also be used in this invention. After distal stent 404 is deployed, a portion of liner 406 is pressed against vessel wall 407 by the deployed stent 404. Then, sheath 408 of catheter 400 is removed to expose the to-be inflated liner 406 and a wire 409 linked to an iliac stent 410 as illustrated in FIG. 16c. Simultaneously, a wire 411 is inserted in aneurysm 401 via left iliac artery 412 to pull wire 409 and iliac stent 410 to the left iliac artery 412 for deployment as shown in FIG. 16d. Distal balloon 405 is then deflated slightly allowing blood flow (as indicated by arrows 414) through space 413 between balloon 405 and distal stent 404 to expand liner 406. Under this hemodynamic pressure, liner 406 expands radially toward aneurysm wall 415 and eventually conforms to the inner surface of aneurysm wall 415 of aneurysm 401 as depicted in FIG. 16e. This procedure is safe because the hemodynamic force to expand liner 406 is no larger than the hemodynamic force before the procedure. No additional stress is placed on aneurysm wall 415 during the expansion of liner 406.

After aneurysm wall 415 is completely covered by liner 406, both iliac stents 410, 416 are deployed in iliac arteries 412, 417 respectively as shown in FIG. 16f. They are used to ensure seal at junctions 418, 419 between liner 406 and iliac arteries 412, 417. Self expandable stents 410, 416 are used herein as an example. Other types of stents such as balloon expandable stents can also be used in this invention. As shown in FIG. 16g, a balloon catheter 420 is inserted in liner 406 via left iliac artery 412. Once it is in position, balloon 421 on the distal end of catheter 420 is inflated with saline. As shown in FIG. 16h, a proximal balloon 422 on delivery catheter 400 is also inflated by saline. Both balloons 421, 422 are used to ensure patency of flow conduit 423 when liner 406 is inflated. The activation of absorbent 424 and inflation of liner 406 gives additional strength to liner 406 and protects aneurysm wall 415 as shown in FIG. 16i. As liner 406 is inflated, the status of inflation is monitored by radiopaque markers 425 on the surface of liner 406. Alternatively, the status of inflation can be observed if absorbent 424 becomes radiopaque when additional radiopaque agent has been added to it. Because outer wall 426 of liner 406 is already conformed to the inner surface of aneurysm wall 415, activated absorbent 424 actually moves inner wall 427 of liner 406 away from ancurysm wall 415. A plurality of connectors 428 between inner wall 427 and outer wall 426 defines the thickness of inflated liner 406. Finally, all balloons 405, 421, 422 are deflated. Both delivery catheter 400 and balloon catheter 420 are retrieved from the patient's body leaving inflated liner 406 in aneurysm 401 as shown in FIG. 16j. This invention is particularly suitable for treating patients with abdominal aortic aneurysms near the iliac bifurcation.

In another embodiment according to the present invention, the liner includes a bioactive or a pharmaceutical agent. The bioactive or pharmaceutical agent can be mixed with absorbent before being encapsulated in the liner. After the deployment, the agent diffuses into the aneurysm wall and treats the disease in the vessel. Because the liner of this invention is in close contact with the aneurysm wall, the agent can reach the aneurysm wall without being diluted by the blood. Dilution decreases the efficacy of the agent when it is delivered orally or by injection. Many bioactive or pharmaceutical agents can be encapsulated in the liner to treat aneurysm in this invention. Agents that inhibit matrix metalloproteinases, inflammation or other pathological processes involved in aneurysm progression, can be incorporated into the liner to enhance wound healing, stabilize and possibly reverse the pathology of aneurysm. Agents that induce positive effects at the aneurysm site, such as growth factor, can also be delivered by the liner and the methods described herein. Exemplary non-limiting examples include platelet-derived growth factor (PDGF), platelet-derived epidermal growth factor (PDEGF), fibroblast growth factor (FGF), transforming growth factor-beta (TGF-β), platelet-derived angiogenesis growth factor (PDAF), transforming growth factor-beta (TGF-β), basic fibroblast growth factor (bFGF), vascular growth factor, vascular endothelial growth factor, and placental growth factor. These agents have been implicated in wound healing by increasing collagen secretion, vascular growth and fibroblast proliferation. Other exemplary non-limiting examples include Doxycycline, Tetracycline, peptides, proteins, hormones, DNA or RNA fragments, genes, cells, cell growth promoting compositions, and autologous platelet gel (APG). Alternatively, the bioactive or pharmaceutical agent can be coated on the outer surface of the liner. The agent or cell growth promoting factor on the outer surface of liner can activate cell growth and proliferation. Those cells adhere to the liner and anchor the liner securely to the vessel lumen and thus preventing migration. Moreover, tissue in-growth on the liner can also provide a seal around the junction of collateral arteries in the aneurysm to prevent endoleak.

In another embodiment of the present invention, the outer wall of the liner is treated to increase its surface area. The increased surface area can increase the contact between the vessel and the liner. Due to the intimate contact with the outer surface of the liner, smooth muscle cells and fibroblasts, etc. in the vessel will be stimulated to proliferate. As these cells proliferate they will grow onto the outer wall of the liner so that the outer wall becomes physically attached to the vessel lumen. The attached cells or tissue on the liner wall can enhance the bonding and seal between the vessel wall and the liner. Increased surface area on the outer wall can further enhance the contact between the vessel and the liner and stimulate more cells proliferate and bonding. In addition, the increase surface area also promotes the formation of thrombosis. The thrombosis can fill gaps between the outer wall of the liner and the surface of the aneurysm wall further preventing endoleak. Typical techniques to increase surface area are sanding, etching, depositing, coating, bonding with fibers or thin foam. Fibers such as PET fibrils are biocompatible with high surface area. They are well-known to the people skilled in the art.

In another embodiment of this invention, a plurality of inflatable liners can be used to treat the aneurysm. Multiple liners form a composite and enhance the overall strength of the liners thus increasing their ability to reduce the hemodynamic pressure on the aneurysm wall. Individual liner can be deployed in the aneurysm sequentially following the methods described in this invention. Or an inflatable liner can comprise more than one layer. Compared with the liner of multiple layers, deploying the liner one layer at a time can reduce the size of the delivery catheter therefore enhancing its ability to maneuver through patient's tortuous iliac anatomy. As a result, each liner can be introduced in the aneurysm with a minimum invasivity. Alternatively, inflated liners of various flexibilities can be used in this invention. For example, the liner with a higher flexibility can be used as the liner adjacent to the inner surface of aneurysm for the optimum conformation to the aneurysm to prevent endoleak. Alternatively, inflated liners of different stiffness can be used in this invention. For example, the liner with a higher stiffness can be used as the liner forming the flow conduit for the enhanced support for the blood flow conduit. Alternatively, inflatable liners of various compositions can be used in this invention. For example, the liner with a higher bioactive agent content can be used as the liner adjacent to the inner surface of aneurysm for the optimum efficacy. Alternatively, inflatable liners of various configurations can be used in this invention. For example, the liner with a higher surface roughness can be used as the liner adjacent to the inner surface of aneurysm for the enhanced ability to promote tissue in-growth and fixation.

In another embodiment of this invention, the inflatable liner comprises a hardening agent such as calcium compound. Calcium phosphate cement (CPC) is biocompatible and has been used as bone cement and dental implant for years. It is consisting of dicalcium phosphate anhydrous (DCPA), CaCO3, Ca(OH)2, alpha-tricalcium phosphate(alpha-TCP), or tetracalcium phosphate (TTCP), etc. The cement will harden when exposed to water and form hydroxyapatite (HA) which is a key component of human bone. In addition to calcium phosphate, other calcium containing compounds can also be used as hardening agent in this invention. Exemplary non-limiting compounds are amorphous calcium phosphate, monocalcium phosphate monohydrate, monocalcium phosphate anhydrous, dicalcium phosphate dehydrate, dicalcium phosphate anhydrous, beta-tricalcium phosphate, octacalcium phosphate, and mixture thereof, etc. Other than calcium phosphate, sodium phosphate and organic acid such as carboxylic acid can be used to accelerate the hardening once the calcium compound is in contact with water. The calcium compound can be mixed with absorbent and encapsulated between the walls by spraying, coating, dipping, etc. The calcium compound/absorbent paste remains flexible in the pliable mode so that the liner remains flexible and can be compressed and loaded in a delivery catheter. After the liner is deployed in the aneurysm, the body fluid permeates through the wall and reacts with the absorbent and calcium compound. Combined with the activated absorbent, the stiffened calcium compound in the liner strengthens the inflated liner, and the liner is thus transformed from the pliable mode to the strengthening mode providing support for the aneurysm wall. These flexible walls on the liner serve as a means not only to contain the calcium compound/absorbent but also control the timing for the calcium compound/absorbent to activate and transform the liner from the pliable mode to the strengthening mode. The permeability of the wall is configured to control the timing for sufficient body fluid to penetrate through the walls and activate the calcium compound/absorbent within the liner.

There are several benefits for this present invention to treat aneurysm. First, the liner can strengthen the aneurysm wall and prevent the rupture of aneurysm by reducing the hemodynamic pressure on the aneurysm wall. Second, the collapsed liner is flexible so that it can be easily loaded in a catheter and access the aneurysm site via iliac artery and then deployed in the aneurysm with minimum invasivity. Third, the flexibility of the liner and the hemodynamic force allow the liner to conform to the inner surface of the aneurysm wall without gap between them. After the absorbent is activated and the liner is inflated, it will be “locked” in the aneurysm without endoleak or migration. Fourth, less material is required to cover the inner surface of aneurysm wall than filling the whole aneurysm. The resulting liner is more flexible than the filler structure that fills the whole aneurysm. This flexible liner is more compatible with the body movement and adjacent organs. Fifth, there is no excess amount of stress on the aneurysm wall during the inflation of the liner. In order to prevent endoleak and migration, it is essential to have close contact between the outer wall of the liner and the surface of the aneurysm wall. To achieve that, the whole aneurysm (other than the tubular flow conduit within the aneurysm) needs to be filled as what was disclosed in the prior arts. Insufficient filler will result in gaps between the liner and the surface of the aneurysm wall. On the other hand, too much filler will place excess circumferential stress on the weak aneurysm wall. However, because the gap and the aneurysm wall have no contrast agent in them and can't be visualized under Fluoroscope, physician cannot determine if the gap has been filled (or not being filled) by the fill structure during the inflation of the fill structure. This uncertainty can place the patient in great risk. Additionally, as described in prior arts, the aneurysm is usually sealed by a stent graft or a lumen shaping balloon before the fill structure is inflated. Existing blood in the aneurysm (with the added filler) can also cause high stress on the aneurysm wall during the inflation of fill structure if the collateral arteries in the aneurysm are occluded. In the present invention, the close contact between the aneurysm wall and the outer wall of the liner is a result of flexible walls and the hemodynamic force. It is not necessary to fill the whole aneurysm in order to close the gap between the aneurysm wall and the liner. As a result, the systems and methods provided by this present invention are safer than what were disclosed in the prior arts. Sixth, the present invention can enhance the adhesion of the liner to the aneurysm wall to further reduce the risk of liner migration and endoleak. Seventh, this invention enables the use of bioactive or pharmaceutical agents in the absorbent to treat aneurysm without dilution. The pathological processes involved in aneurysm progression can be stabilized and possibly be reversed. Eighth, the flexible liner does not have the issue of kinking or occlusion of blood flow which is common in tubular stent graft. Ninth, the durability of the liner is better than the tubular stent graft because there is no untreated space, which is prone to endoleak, between the liner and aneurysm wall.

Those skilled in the art will further appreciate that the embodiments according to the teaching of present invention may include other specific forms or characteristics without departing from the spirit of this invention thereof. The present invention is not limited in the particular embodiments described in detail therein. The foregoing description discloses only exemplary embodiments, other variations are considered as being within the scope of the present invention. Numerous references cited herein are incorporated by reference in their entirety.

I claim:

1. A system to protect the wall of an aneurysm in a vessel wherein the system comprises:
a liner comprising one or more inflatable chambers, said one or more inflatable chambers comprising one or more connectors and absorbent, wherein said liner is configured to conform to the interior surface of the aneurysm following introduction of said liner into the vessel, and wherein said one or more connectors constrains expansion of said one or more chambers upon inflation of said absorbent.

2. The system as set forth in claim 1 further comprising means for anchoring said liner to the interior of the vessel.

3. The system as set forth in claim 2, wherein said means for anchoring said liner comprises one or more expandable elements coupled to said liner.

4. The system as set forth in claim 3, wherein said one or more expandable elements comprises a stent.

5. The system as set forth in claim 1, wherein one or more of said inflatable chambers comprises one or more opposing interior walls and said one or more connectors is affixed to opposing interior walls, wherein said one or more opposing interior walls are permeable to body fluid.

6. The system as set forth in claim 5, wherein said one or more connectors comprises a strip, a string, or a bond.

7. The system as set forth in claim 1, wherein said one or more inflatable chambers comprises an inflatable patch or an inflatable channel.

8. The system as set forth in claim 1, wherein said one or more inflatable chambers is disposed helically on the exterior of said liner.

9. The system as set forth in claim 1, wherein said one or more inflatable chambers is disposed circumferentially on the exterior of said liner.

10. The system as set forth in claim 1, wherein the said liner comprises flexible and substantially inelastic biocompatible material.

11. The system as set forth in claim 1, wherein said liner comprises an inner wall defining a main flow conduit of the vessel proximate the aneurysm following introduction of the liner into the vessel, said conduit comprising an inlet and one or more outlets.

12. The system as set forth in claim 11, wherein said main flow conduit is defined by the inner surface of the aneurysm, said connectors and the amount absorbent in said liner.

13. The system as set forth in claim 1, wherein said absorbent comprises a hydrogel or a hydrophilic material.

14. The system as set forth in claim 1, wherein said absorbent is selected from the group consisting of polymethacrylic acid, polyacrylic acid, polyesters, polyacrylamide, polyacrylamide copolymer, sodium acrylate and vinyl alcohol copolymer, polyvinyl alcohol, polyacetals, polyvinyl acetate, acrylic acid ester copolymer, polyvinyl pyrrolidone, polyacrylonitrile, polyarylethernitriles, Hypan, poly(2-hydroxyethyl methacrylate)(polyHEMA), Carbomer copolymer and homopolymer, alkoxylated surfactants, polyethylene oxide, poly(propylene oxide), poly(ethylene glycol), poly(propylene glycol), poly(vinylcarboxylic acid), collagen, polyvinyl pyridine, polylysine, polyarginine, poly aspartic acid, poly glutamic acid, polytetramethylene oxide, methoxylated pectin gels, cellulose acetate phthalate, gelatin, alginate, calcium alginate, Carbopol, Poloxamer, Pluronic, Tetronics, PEO-PPO-PEO triblocks copolymer, Tetrafunctional block copolymer of PEO-PPO condensed with ethylenadiamine, Poly(acrylic acid) grafted (PEO-PPO-PEO-PAA) copolymers, graft copolymers of Pluronic and poly(acrylic acid), alkylcellulose, hydroxyalkylcellulose, PEG-PLA-PEG block polymers, Poly(N-isopropylacrylamide) (PNIPAAm), tetrafunctional block copolymer of PEO-PPO-ethylenadiamine, copolymer of PNIPAAm and acrylic acid (AAc), P(NIPAAm-co-AAc), copovidone, povidone, Hyaluronic Acid (HA), polyoxyalkylene ether, cellulose acetate, cellulose, cellulose diacetate, nitrocellulose, starch, and copolymer and mixture thereof.

15. The system as set forth in claim 1, wherein said absorbent is activated by body fluid, wherein said body fluid enables said absorbent to expand and increase the thickness of said liner.

16. The system as set forth in claim 1, further comprising a hardening agent encapsulated in said liner, wherein the hardening agent is hardened by the body fluid.

17. The system as set forth in claim 1 wherein said absorbent comprises a bioactive or a pharmaceutical active component.

18. The system as set forth in claim 1, wherein the liner comprises an outer surface comprising a bioactive or a pharmaceutical active component.

19. The system as set forth in claim 1, wherein the liner comprises an outer surface and surface area, wherein said outer surface is treated with fibers, fibril, foam, or roughening to increase the surface area.

20. The system as set forth in claim 1 further comprising means to introduce a hemodynamic force in said liner whereby said liner expands and conforms to the interior surface of the aneurysm.

21. An endovascular system to protect aneurysm wall comprising:
an inflatable liner having an inner wall and an outer wall and an absorbent encapsulated between walls, said inflatable liner having an inlet and an outlet and a dimension no less than the lumen of the aneurysm, said inflatable liner being collapsible in the delivery system and expandable to conform to said blood vessel, said inflatable liner having at least one wall being permeable to body fluid, said absorbent being activated by said body fluid to expand and increase the thickness of said inflatable liner, said inflated liner providing support to said blood vessel.

22. The system as set forth in claim 21 further comprising means for anchoring said liner to the interior of the vessel.

23. The system as set forth in claim 22, wherein said means for anchoring said liner comprises one or more expandable elements coupled to said liner.

24. A method of treatment of an aneurysm comprising:
providing an inflatable liner comprising one or more inflatable chambers and absorbent; and
anchoring a portion of the inflatable liner in the vessel adjacent the aneurysm with a first expandable element; and
introducing hemodynamic force in the inflatable liner whereby said liner expands and conforms to the interior surface of the aneurysm.
introducing body fluid into the one or more inflatable chambers whereby said absorbent expands said one or more inflatable chambers and protects the vessel.
